(19) United States
(12) Patent Application Publication
(10) Pub. No.: US 2026/0008819 A1
(43) Pub. Date: Jan. 8, 2026

(54) CYCLOSPORINE-ACRIDINIUM ESTERS AND METHODS OF PRODUCTION AND USE THEREOF

(71) Applicant: Siemens Healthcare Diagnostics Inc., Tarrytown, NY (US)

(72) Inventors: Zhijian Zhao, Wilmington, DE (US); Bogdan Draghici, Newark, DE (US); Yi Feng Zheng, Wilmington, DE (US); Michele Smith, Poughkeepsie, NY (US); SunRay DiFrancesco, Peekskill, NY (US); Jane Zhang, New York, NY (US); Debra Hovanec-Burns, Wappingers Falls, NY (US); William Bedzyk, Odessa, DE (US)

(73) Assignee: Siemens Healthcare Diagnostics Inc., Tarrytown, NY (US)

(21) Appl. No.: 19/136,678

(22) PCT Filed: Oct. 24, 2023
(86) PCT No.: PCT/US2023/077602

§ 371 Date: Jun. 6, 2025

Related U.S. Application Data
(60) Provisional application No. 63/476,552, filed on Dec. 21, 2022.

(51) Int. Cl. C07K 7/64; G01N 33/543

(52) U.S. Cl. CPC C07K 7/645; G01N 33/543

(57) ABSTRACT

Compositions are disclosed that include acridinium esters of Cyclosporine A or C. Also disclosed are kits containing same and methods of producing and using same.

### CROSS REFERENCE TO RELATED APPLICATIONS/INCORPORATION BY REFERENCE STATEMENT

The subject application claims benefit under 35 USC § 119 (e) of U.S. Provisional Application No. 63/476,552, filed Dec. 21, 2022. The entire contents of the above-referenced patent application(s) are hereby expressly incorporated herein by reference.

### STATEMENT REGARDING FEDERALLY SPONSORED RESEARCH OR DEVELOPMENT

Not Applicable.

### BACKGROUND

The body relies upon a complex immune response system to distinguish self from non-self. At times, the body's immune system must be controlled in order to either augment a deficient response or suppress an excessive response. For example, when organs such as (but not limited to) kidney, heart, heart-lung, bone marrow, and liver are transplanted in humans, the body will often reject the transplanted tissue by a process referred to as allograft rejection.

In treating allograft rejection, the immune system is frequently suppressed in a controlled manner with drug therapy. Immunosuppressant drugs are carefully administered to transplant recipients in order to help prevent allograft rejection of non-self tissue. Some of the most commonly administered immunosuppressive drugs to prevent organ rejection in transplant patients are Cyclosporine A (CsA), Mycophenolic acid, FK-506 (also known as tacrolimus), sirolimus (also known as rapamycin), and everolimus.

The side effects associated with immunosuppressant drugs can be controlled in part by carefully controlling the level of the drug present in a patient. Therapeutic monitoring of concentrations of immunosuppressant drugs and related drugs in blood is required to optimize dosing regimens to ensure maximal immunosuppression with minimal toxicity. Although immunosuppressant drugs are highly effective immunosuppressive agents, their use must be carefully managed, because the effective dose range is often narrow, and excessive dosage can result in serious side effects. On the other hand, too low of a dosage of an immunosuppressant can lead to tissue rejection. Because the distribution and metabolism of an immunosuppressant drug can vary greatly between patients, and because of the wide range and severity of adverse reactions, accurate monitoring of the drug level is essential.

There is, therefore, a continuing need to develop fast and accurate diagnostic methods to measure levels of analytes (such as immunosuppressant drugs like CsA) in samples taken from a patient. The methods should be fully automatable and be accurate even when conducted on samples having various interfering substances present. The assay should provide an accurate measurement of the amount of the analyte in the sample, while minimizing inaccuracies resulting from interfering substances present in the sample.

### BRIEF DESCRIPTION OF THE DRAWINGS

FIG. 1 depicts the chemical structures of Cyclosporine A (CsA) and Cyclosporine C (CsC).

FIG. 2 depicts the chemical structure of the prior art Cyclosporine C-acridinium ester (CsC AE) tracer CsC-NSP-DMAE-HEG3-CsA (C₁₃₈H₂₂₆N₁₈O₄₀S; Mol. Wt 2809.43).

FIG. 3 graphically depicts one non-limiting embodiment of Cyclosporine A assay format constructed in accordance with the present disclosure.

FIG. 4 depicts the chemical structure of one non-limiting embodiment of a composition constructed in accordance with the present disclosure and labeled as CsC-DA-10-NSP-DMAE (Formula VI).

FIG. 5 depicts the chemical structure of one non-limiting embodiment of a composition constructed in accordance with the present disclosure and labeled as CsC-DA-10-TSPAE (Formula VII).

FIG. 6 depicts the chemical structure of one non-limiting embodiment of a composition constructed in accordance with the present disclosure and labeled as CsC-DA-10-HEGAE (Formula VIII).

FIG. 7 depicts the chemical structure of one non-limiting embodiment of a composition constructed in accordance with the present disclosure and labeled as CsC-DA-10-NSP-ZAE (Formula IX).

FIG. 8 depicts the chemical structure of one non-limiting embodiment of a composition constructed in accordance with the present disclosure and labeled as CsC-PEG15-ZAE (Formula X).

FIG. 9 depicts the chemical structure of one non-limiting embodiment of a composition constructed in accordance with the present disclosure and labeled as CsC-PEG23-ZAE (Formula XI).

FIG. 10 depicts the chemical structure of one non-limiting embodiment of a composition constructed in accordance with the present disclosure and labeled as CsC-PEG23-NSP-DMAE (Formula XII).

FIG. 11 depicts the chemical structure of one non-limiting embodiment of a composition constructed in accordance with the present disclosure and labeled as CsA-Oxime-PEG3-HEGAE (Formula XIII).

FIG. 12 depicts the chemical structure of one non-limiting embodiment of a composition constructed in accordance with the present disclosure and labeled as CsA-Oxime-PEG3-ZAE (Formula XIV).

FIG. 13 depicts the chemical structure of one non-limiting embodiment of a composition constructed in accordance with the present disclosure and labeled as CsA-Pent-DA-10-NSP-DMAE (Formula XV).

FIG. 14 depicts the chemical structure of one non-limiting embodiment of a composition constructed in accordance with the present disclosure and labeled as CsA-Pent-DA-10-ZAE (Formula XVI).

FIG. 15 depicts the chemical structure of one non-limiting embodiment of a composition constructed in accordance with the present disclosure and labeled as CsA-Pent-DA-10-FG-DA13-ZAE (Formula XVII).

FIG. 16 graphically depicts a synthesis scheme for CsC-DA-10-NSP-DMAE of FIG. 4.

FIG. 17 graphically depicts a synthesis scheme for CsC-DA-10-TSPAE of FIG. 5.

FIG. 18 graphically depicts a synthesis scheme for CsC-DA-10-HEGAE of FIG. 6.

FIG. 19 graphically depicts a synthesis scheme for CsC-DA-10-NSP-ZAE of FIG. 7.

FIG. 20 graphically depicts a synthesis scheme for CsC-PEG15-ZAE of FIG. 8.

FIG. 21 graphically depicts a synthesis scheme for CsC-PEG23-ZAE of FIG. 9.

FIG. 22 graphically depicts a synthesis scheme for CsC-PEG23-NSP-DMAE of FIG. 10.

FIG. 23 graphically depicts a synthesis scheme for CsA-Oxime-PEG3-HEGAE of FIG. 11.

FIG. 24 graphically depicts a synthesis scheme for CsA-Oxime-PEG3-ZAP of FIG. 12.

FIG. 25 graphically depicts a synthesis scheme for CsA-Pent-DA-10-NSP-DMAE of FIG. 13.

FIG. 26 graphically depicts a synthesis scheme for CsA-Pent-DA-10-ZAE of FIG. 14.

FIG. 27 graphically depicts a synthesis scheme for CsA-Pent-DA-10-HG-DA13-ZAE of FIG. 15.

FIG. 28 illustrates acridinium ester (AE) screening using CsA-HEG3-NSP-DMAE, CsC-NSP-DA-10-NSP-DMAE, CSC-DA-10-NSP-ZAE, CSC-DA-10-TSPAE, and CsC-DA-10-HEGAE in a non-preformed CsA assay format.

FIG. 29 graphically depicts binding curves for the Cyclosporine C-Acridinium Esters (CsC-AEs) of HEG3, NSP-DMAE, NSP-ZAE, TSP-AE, and HEGAE in a non-preformed CsA assay format.

FIG. 30 illustrates AE screening using CsC-DA-10-NSP-ZAE, CsC-O-PEG15-ZAE, CsC-PEG23, ZAE, and CsC-PEG23-NSP-DMAE in a non-preformed CsA assay format.

FIG. 31 graphically depicts binding curves for the CsC-AEs of -DA-10-NSP-ZAE, -PEG15-ZAE, -PEG23-ZAE, and -PEG23-NSP-DMAE in a non-preformed CsA assay format.

FIG. 32 illustrates AE screening using CsA-Oxime-PEG3-ZAE, CsA-Pent-DA-10-NSP-DMAE, CsA-Pent-DA-10-ZAE, and CsA-Pent-DA-10-HG-DA-13-ZAE in a non-preformed CsA assay format.

FIG. 33 graphically depicts binding curves for the Cyclosporine A-Acridinium Esters (CsA-AEs) of -Oxime-PEG3-ZAE, -NSP-DMAE, -NSP-ZAE, and -HG-DA-13-ZAE in a non-preformed CsA assay format.

FIG. 34 illustrates an Ambient temperature effort (ATE) screening study using CsC AEs (HEG3, DMAE, NSP-ZAE, TSPAE, and HEGAE).

FIG. 35 illustrates an ATE study using CsC-DA-10-NSP-ZAE, CsC-PEG23-NSP-DMAE, CsC-PEG15-ZAE, CsC-PEG23-ZAE, and CsA-Pent-ZAE.

### DETAILED DESCRIPTION

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles, compositions, kits, and/or methods disclosed herein can be made and executed without undue experimentation in light of the present disclosure. While the articles, compositions, kits, and/or methods have been described in terms of particular embodiments, it will be apparent to those of skill in the art that variations may be applied to the articles, compositions, kits, and/or methods and in the steps or in the sequence of steps of the methods described herein without departing from the concept, spirit, and scope of the present disclosure. All such similar substitutes and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The terms “analog” and “derivative” are used herein interchangeably and refer to a substance which comprises the same basic carbon skeleton and carbon functionality in its structure as a given compound, but can also contain one or more substitutions thereto. The term “substitution” as used herein will be understood to refer to the replacement of at least one substituent on a compound with a residue R. In certain non-limiting embodiments, R may include H, hydroxyl, thiol, a halide selected from fluoride, chloride, bromide, or iodide, a C1-C4 compound selected one of the following: linear, branched or cyclic alkyl, optionally substituted, and linear branched or cyclic alkenyl, wherein the optional substituents are selected from one or more alkenylalkyl, alkynylalkyl, cycloalkyl, cycloalkenylalkyl, arylalkyl, heteroarylalkyl, heterocyclealkyl, optionally substituted heterocycloalkenylalkyl, arylcycloalkyl, and arylheterocycloalkyl, each of which is optionally substituted wherein the optional substituents are selected from one or more of alkenylalkyl, alkynylalkyl, cycloalkyl, cycloalkenylalkyl, arylalkyl, alkylaryl, heteroarylalkyl, heterocyclealkyl, optionally substituted heterocycloalkenylalkyl, arylcycloalkyl, and arylheterocyclalkyl, phenyl, cyano, hydroxyl, alkyl, aryl, cycloalkyl, cyano, alkoxy, alkylthio, amino, —NH (alkyl), —NH (cycloalkyl) 2, carboxy, and —C(O))-alkyl.

The term “sample” as used herein will be understood to include any type of biological sample that may be utilized in accordance with the present disclosure. Examples of fluidic biological samples that may be utilized include, but are not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, combinations thereof, and the like.

The term “specific binding partner” or “analyte-specific binder” will be understood to refer to any molecule capable of specifically associating with a target analyte. For example, but not by way of limitation, the binder/binding partner may be an antibody, a receptor, a ligand, aptamers, molecular imprinted polymers (i.e., inorganic matrices), any fragments thereof, and any combinations or derivatives thereof, as well as any other molecules capable of specific binding to the target analyte.

The term “antibody” is used in the broadest sense, and specifically (but not by way of limitation) covers monoclonal antibodies (including full length monoclonal antibodies), polyclonal antibodies, multispecific antibodies (e.g., bispecific antibodies), fragments of any of the above, and conjugates of any of the above, so long as they exhibit the desired biological activity of analyte binding. Thus, the term “antibody” or “antibody peptide(s)” refers to a full-length immunoglobulin molecule (i.e., an intact antibody) or an antigen-binding fragment thereof that competes with the intact antibody for specific antigen binding. Antigen-binding fragments may be produced by recombinant DNA techniques, or by enzymatic or chemical cleavage of intact antibodies. Antigen-binding fragments include Fab, Fab′, F(ab′)₂, Fv, scFv, disulfide linked Fv, Fd, diabodies, single-chain antibodies, single domain antibodies (such as but not limited to, NANOBODIES®), and other antibody fragments or conjugates thereof that retain at least a portion of the variable region of an intact antibody, antibody substitute proteins or peptides (i.e., engineered binding proteins/peptides), and combinations or derivatives thereof. See, e.g., Hudson et al. (Nature Med. (2003) 9:129-134). The antibody can be of any type or class (e.g., IgG, IgE, IgM, IgD, and IgA) or sub-class (e.g., IgG1, IgG2, IgG3, IgG4, IgA1, and IgA2).

The term “antigen binding fragment” or “antigen-binding portion” of an antibody, as used herein, refers to one or more fragments of an antibody that retain the ability to bind to an antigen. The antigen-binding function of an antibody can be performed by fragments of an intact antibody. Examples of binding fragments encompassed within the term “antigen-binding fragment” of an antibody include but are not limited to, Fab, Fab′, F(ab′)₂, Fv, scFv, disulfide linked Fv, Fd, diabodies, single-chain antibodies, single domain antibodies (such as but not limited to, NANOBODIES®), isolated CDRH3, and other antibody fragments that retain at least a portion of the variable region of an intact antibody. These antibody fragments are obtained using conventional recombinant and/or enzymatic techniques and are screened for antigen binding in the same manner as intact antibodies.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. Kappa and lambda light chains refer to the two major antibody light chain isotypes.

The terms “CDR,” and its plural “CDRs,” refer to a complementarity determining region (CDR) of an antibody or antibody fragment, which determine the binding character of an antibody or antibody fragment. In most instances, three CDRs are present in a light chain variable region (CDRL1, CDRL2 and CDRL3) and three CDRs are present in a heavy chain variable region (CDRH1, CDRH2 and CDRH3). CDRs contribute to the functional activity of an antibody molecule and are separated by amino acid sequences that comprise scaffolding or framework regions. Among the various CDRs, the CDR3 sequences, and particularly CDRH3, are the most diverse and therefore have the strongest contribution to antibody specificity. There are at least two techniques for determining CDRs: (1) an approach based on cross-species sequence variability (i.e., Kabat et al., Sequences of Proteins of Immunological Interest (National Institute of Health, Bethesda, Md. (1987), incorporated by reference in its entirety); and (2) an approach based on crystallographic studies of antigen-antibody complexes (Chothia et al., Nature, 342:877 (1989), incorporated by reference in its entirety).

The term “epitope” includes any protein determinant capable of specific binding to an immunoglobulin or T-cell receptor. In certain embodiments, an epitope is a region of an antigen that is specifically bound by an antibody. Epitopic determinants usually include chemically active surface groupings of molecules such as amino acids, sugar side chains, phosphoryl, or sulfonyl groups. In certain embodiments, an epitope may have specific three-dimensional structural characteristics (e.g., a “conformational epitope”), as well as specific charge characteristics.

An epitope is defined as “the same” as another epitope if a particular antibody specifically binds to both epitopes. In certain embodiments, polypeptides having different primary amino acid sequences may comprise epitopes that are the same. In certain embodiments, epitopes that are the same may have different primary amino acid sequences. Different antibodies are said to bind to the same epitope if they compete for specific binding to that epitope.

An antibody “specifically binds” an antigen when it preferentially recognizes the antigen in a complex mixture of proteins and/or macromolecules. In certain embodiments, an antibody comprises an antigen-binding site that specifically binds to a particular epitope. In certain such embodiments, the antibody is capable of binding different antigens so long as the different antigens comprise that particular epitope or closely related epitopes. In certain instances, for example, homologous proteins from different species may comprise the same epitope. In certain embodiments, an antibody specifically binds to an antigen with a dissociation constant of no greater than 10⁻⁶ M, 10⁻⁷ M, 10⁻⁸ M or 10⁻⁹ M. When an antibody specifically binds to a receptor or ligand (i.e., counterreceptor), it may substantially inhibit adhesion of the receptor to the ligand. As used herein, an antibody substantially inhibits adhesion of a receptor to a ligand when an excess of antibody reduces the quantity of receptor bound to ligand by at least about 20%, 40%, 60% or 80%, 85%, or 90% (as measured in an in vitro competitive binding assay).

An “isolated” antibody is one which has been separated and/or recovered from a component of the environment in which it was produced. Contaminant components of its production environment are materials which would interfere with diagnostic or therapeutic uses for the antibody, and may include enzymes, hormones, and other proteinaceous or non-proteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequenator, such as at least 15 residues of sequence; or 3) to homogeneity by SDS-PAGE under reducing or non-reducing conditions using Coomassie blue or, alternatively, silver stain. Isolated antibody includes the antibody in situ within recombinant cells since at least one component of the environment in which the antibody is produced will not be present. Ordinarily, however, isolated antibody will be prepared by at least one purification step. In addition, the “isolated antibody” is substantially free of other antibodies having different antigenic specificities. An isolated antibody may, however, have some cross-reactivity to other, related antigens.

The term “antibody mutant” refers to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, or at least 95%.

The term “monoclonal antibody” as used herein refers to an antibody obtained from a population of substantially homogeneous antibodies that specifically bind to the same epitope, i.e., the individual antibodies comprising the population are identical except for possible naturally occurring mutations that may be present in minor amounts. In contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that in one method of production they may be synthesized by a hybridoma culture, and thus are uncontaminated by other immunoglobulins. The modifier “monoclonal” indicates the character of the antibody as being obtained from a substantially homogeneous population of antibodies, and is not to be construed as requiring production of the antibody by any particular method. For example, in one embodiment, the monoclonal antibodies produced in accordance with the present disclosure may be made by the hybridoma method first described by Kohler and Milstein (Nature, 256:495 (1975)).

The monoclonal antibodies utilized in accordance with the present disclosure may be produced by any methodology known in the art including, but not limited to, a result of a deliberate immunization protocol; a result of an immune response that results in the production of antibodies naturally in the course of a disease or cancer; phage-derived antibodies; and the like. In addition to the hybridoma production method listed above, the monoclonal antibodies of the present disclosure may be produced by other various methods such as, but not limited to, recombinant DNA methods (see, e.g., U.S. Pat. No. 4,816,567); isolation of antibody fragments from a phage display library (see, e.g., Clackson et al., Nature (1991) 352:624-628; and Marks et al., J. Mol. Biol. (1991) 222:581-597); as well as various other monoclonal antibody production techniques (see, e.g., Harlow and Lane (1988) Antibodies: A Laboratory Manual (Cold Spring Harbor Laboratory, Cold Spring Harbor, N.Y.)). Further, many monoclonal antibodies that may be utilized in the conjugates and methods disclosed or otherwise contemplated herein are widely commercially available, and therefore no further description thereof is deemed necessary.

As used herein, “substantially pure” means an object species is the predominant species present (i.e., on a molar basis it is more abundant than any other individual species in the composition). Generally, a substantially pure composition will comprise more than about 50% percent of all macromolecular species present in the composition, such as more than about 55%, 60%, 65%, 70%, 75%, 80%, 85%, 90%, 95%, and 99%. In one embodiment, the object species is purified to essential homogeneity (contaminant species cannot be detected in the composition by conventional detection methods) wherein the composition consists essentially of a single macromolecular species.

An “analyte” is a molecule that is capable of being recognized by an analyte-specific binding partner, such as (but not limited to) an antibody. An analyte comprises at least one antigenic determinant or “epitope,” which is the region of the analyte which binds to the analyte-specific binding partner (i.e., antibody).

Certain non-limiting embodiments of the present disclosure are directed to a composition that comprises a cyclosporine C and an acridinium ester linked via a spacer and having the structure of Formula I:

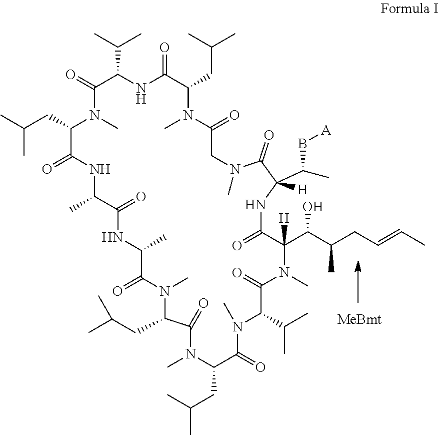

and/or a composition that comprises a cyclosporine A and an acridinium ester (A) linked via a spacer (B) and having the structure of Formula II:

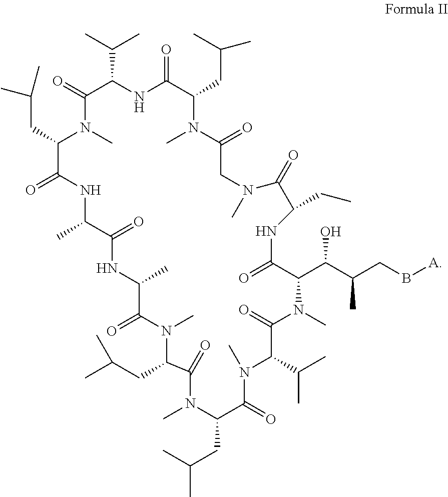

In each of Formulas I and II, “A” comprises an acridinium ester, and “B” is a spacer having from about 5 atoms to about 100 atoms, each selected from the group consisting of C, H, O, N, S, and P atoms.

In particular non-limiting embodiments, the acridinium ester utilized in accordance with the present disclosure has the structure of Formula III:

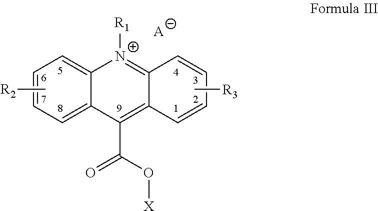

wherein “R1” is selected from the group consisting of an alkyl, alkenyl, alkynyl, or aralkyl group of 1 to 35 carbon atoms and 0 to 20 heteroatoms; a sulfopropyl or sulfobutyl group; and a group —R^{a}—Z, where R^{a} is a divalent radical selected from alkyl, alkenyl, alkynyl, aryl, or aralkyl group of 1 to 35 carbon atoms and 0 to 20 heteroatoms. “R2” is placed at one or more of positions C1 to C4, and “R3” is placed at one or more of positions C5 to C8. Each “R2” and “R3” is independently selected from the group consisting of hydrogen, alkyl, OR, OH, SR, SH, NH₂, and NR′R″, wherein R, R′, and R″ are each independently selected from the group consisting of an alkyl, alkenyl, alkynyl, aryl, and aralkyl group, wherein each group contains 0 to 20 heteroatoms. Also, “X” is a group selected from a halogenated or unhalogenated, branched or straight-chained alkyl group; a substituted or unsubstituted aryl group; and a heterocyclic ring group. The “X” group also comprises 0 to 20 heteroatoms, and further comprises a functional group that links to the spacer “B” of Formula I. In addition, “A⁻” is a counter ion introduced, for example (but not by way of limitation) to pair with the quaternary nitrogen of said acridinium nucleus, and “A⁻” is selected from the group consisting of CH₃SO₃⁻, FSO₃⁻, CF₃SO₃⁻, C₄F₉SO₃⁻, CH₃C₆H₄SO₃⁻, a halide, CF₃COO⁻, CH₃COO⁻, and NO₃⁻.

In particular non-limiting embodiments, the acridinium ester utilized in accordance with the present disclosure is a chemiluminescent acridinium ester having the structure of Formula IV:

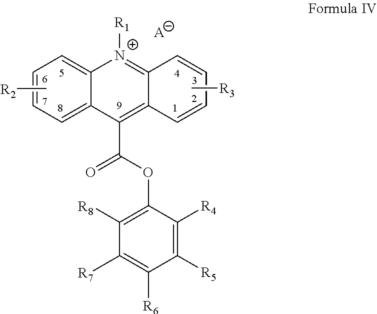

wherein: “R₁,” “R₂,” “R₃,” and “A” are as defined above in reference to Formula III; each of “R₄” and “R₈” is independently selected from hydrogen or an alkyl, alkenyl, alkynyl, alkoxyl (—OR), alkylthiol (—SR), or substituted amino group that serve, for example (but not by way of limitation) to stabilize the -COX- linkage between the acridinium nucleus and the “Y” moiety through steric and/or electronic effect. In addition, each of “R₅,” “R₆,” and “R₇” is independently selected from hydrogen or an alkyl, alkenyl, alkynyl, aryl, or aralkyl group, wherein each group contains 0 to 20 heteroatoms. Also, one of “R₅,” “R₆,” and “R₇” further comprises a functional group that links to the spacer “B” of Formula I. Functional groups that may be utilized in accordance with the present disclosure include, but are not limited to, the following groups:

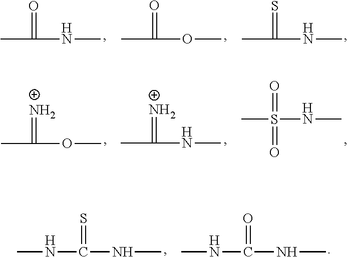

In particular non-limiting embodiments, the acridinium ester utilized in accordance with the present disclosure is a dimethylphenyl acridinium ester having the structure of Formula V:

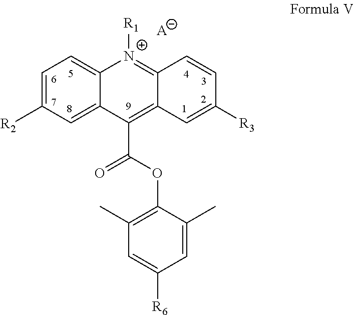

wherein “R₁” is a methyl or a sulfopropyl group; each of “R₂” and “R₃” is independently selected from hydrogen or a methoxy, sulfopropyloxyl, or poly(ethylene)glycoloxy group; and “R₆” is an amide group (CONH—) connecting to the spacer “B” of Formula I. “A” is as defined in Formula III.

In certain particular (but non-limiting) embodiments, R₁ is a sulfopropyl group, and wherein each of R₂ and R₃ is a hydrogen or a sulfopropyloxyl group.

In a particular (but non-limiting) embodiment, the composition is a Cyclosporine C-Acridinium Ester having the structure of Formula VI:

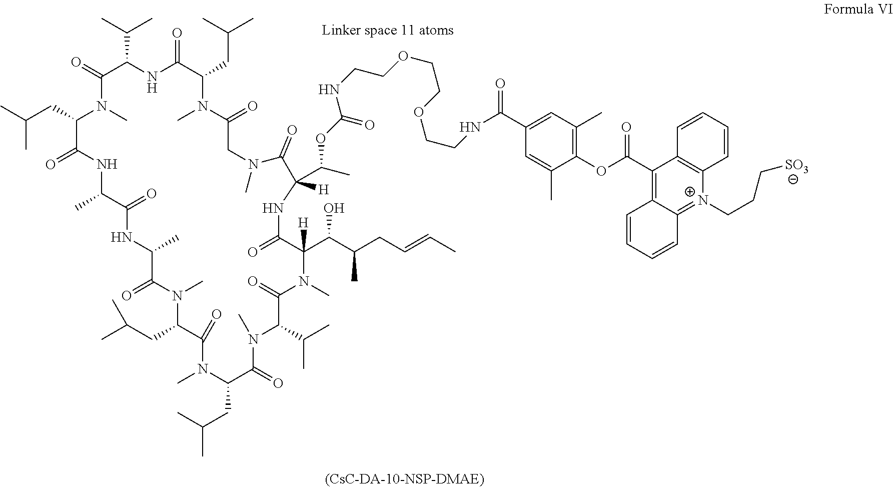

In a particular (but non-limiting) embodiment, the composition is a Cyclosporine C-Acridinium Ester having the structure of Formula VII:

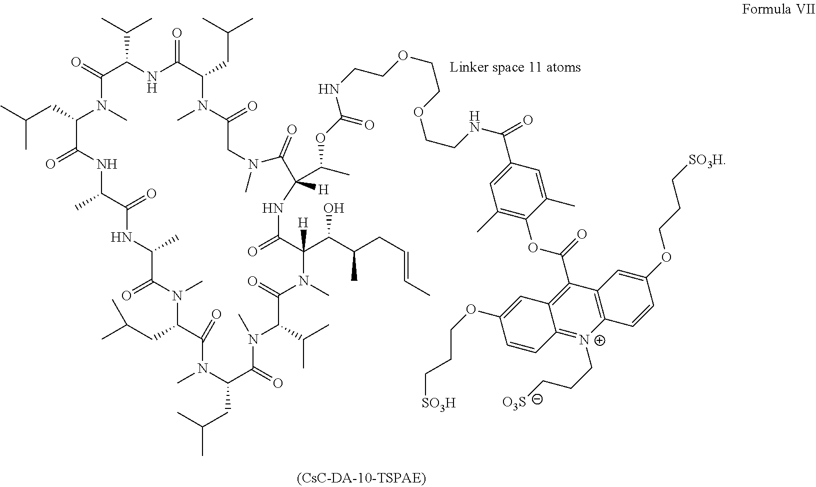

In a particular (but non-limiting) embodiment, the composition is a Cyclosporine C-Acridinium Ester having the structure of Formula VIII:

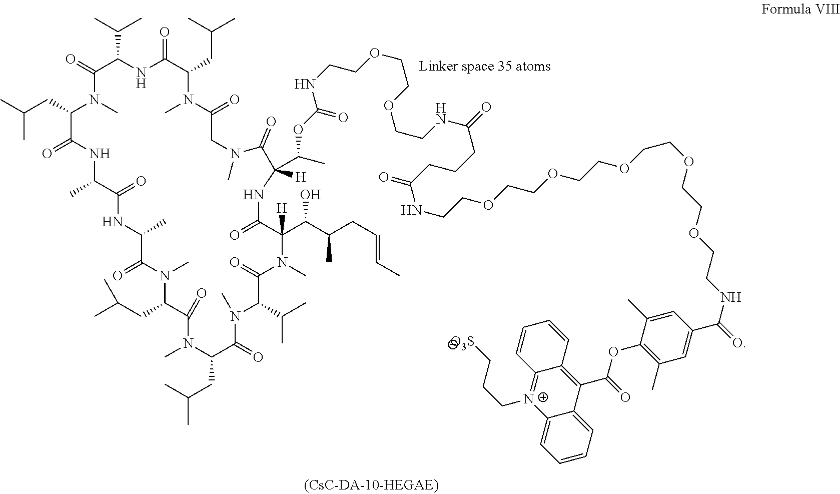

In a particular (but non-limiting) embodiment, the composition is a Cyclosporine C-Acridinium Ester having the structure of Formula IX:

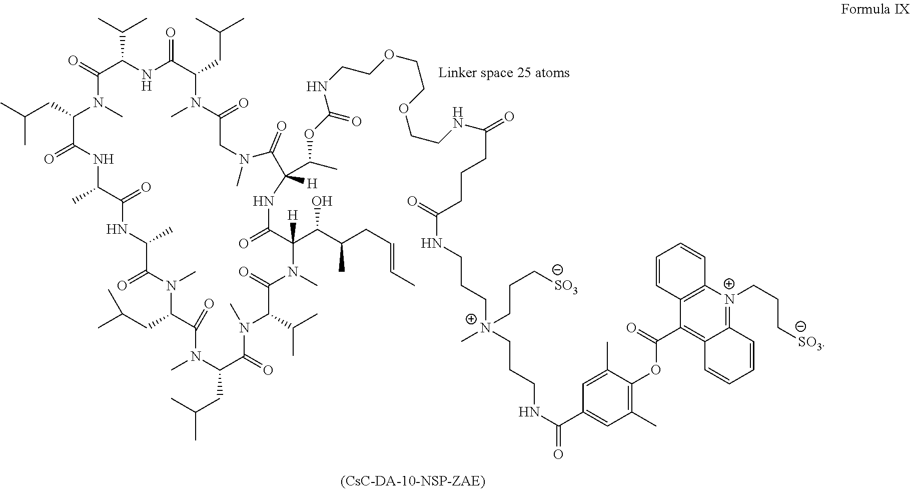

In a particular (but non-limiting) embodiment, the composition is a Cyclosporine C-Acridinium Ester having the structure of Formula X:

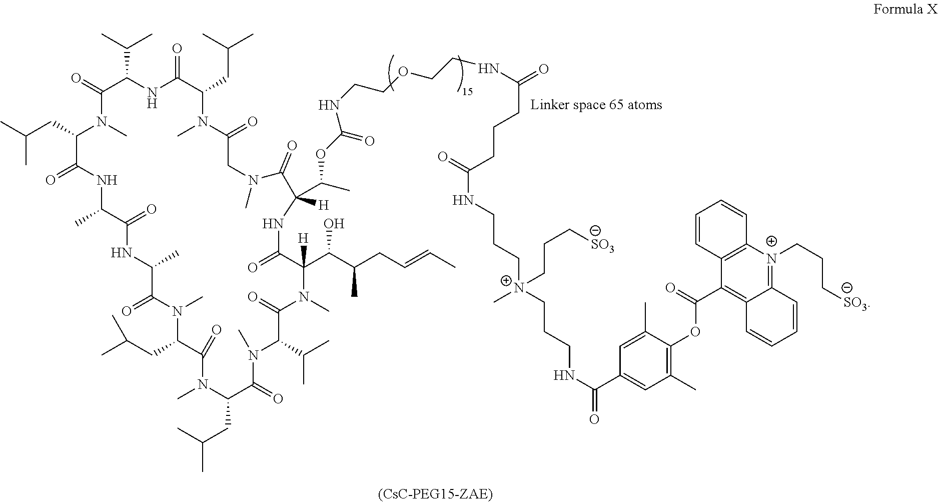

In a particular (but non-limiting) embodiment, the composition is a Cyclosporine C-Acridinium Ester having the structure of Formula XI:

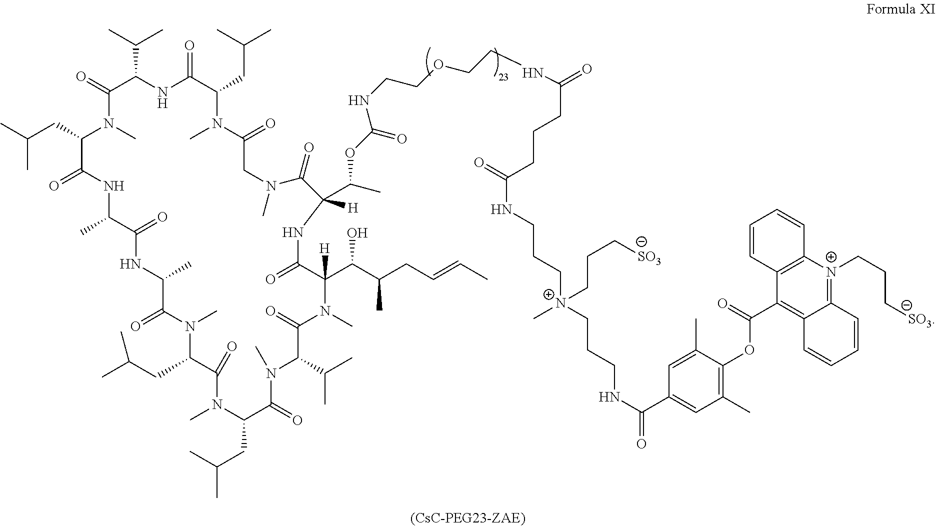

In a particular (but non-limiting) embodiment, the composition is a Cyclosporine C-Acridinium Ester having the structure of Formula XII:

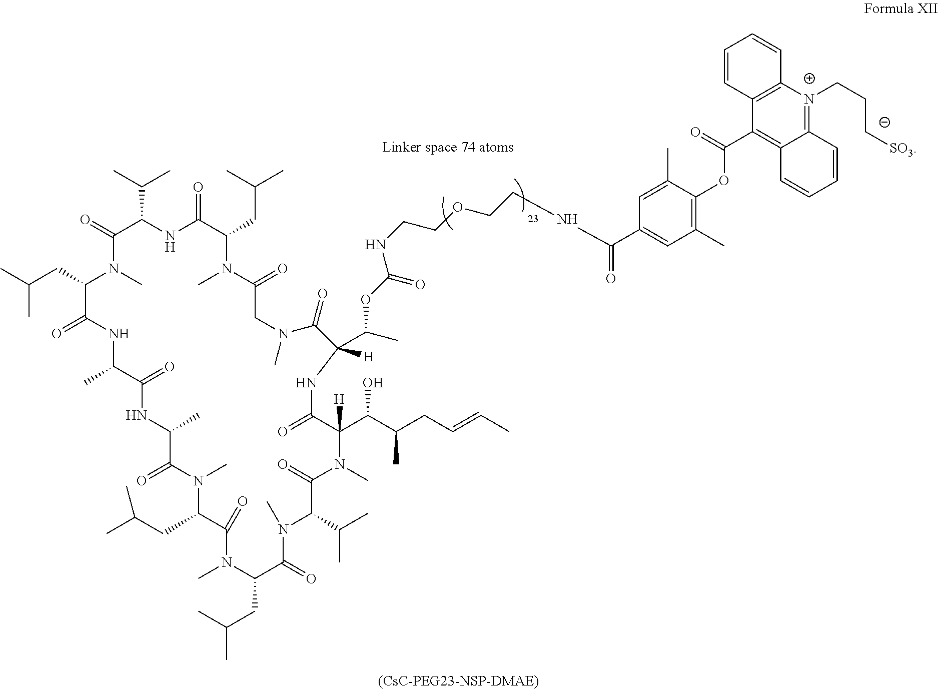

In a particular (but non-limiting) embodiment, the composition is a Cyclosporine A-Acridinium Ester having the structure of Formula XIII:

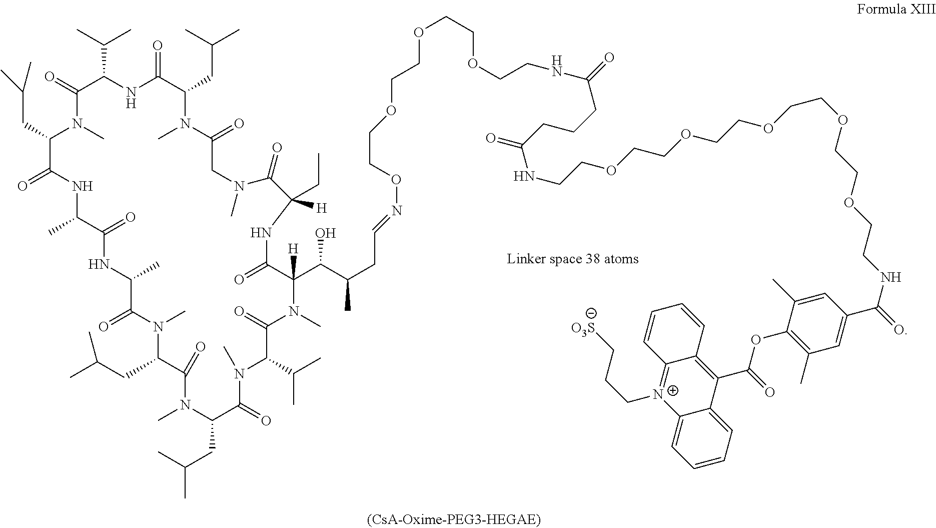

In a particular (but non-limiting) embodiment, the composition is a Cyclosporine A-Acridinium Ester having the structure of Formula XIV:

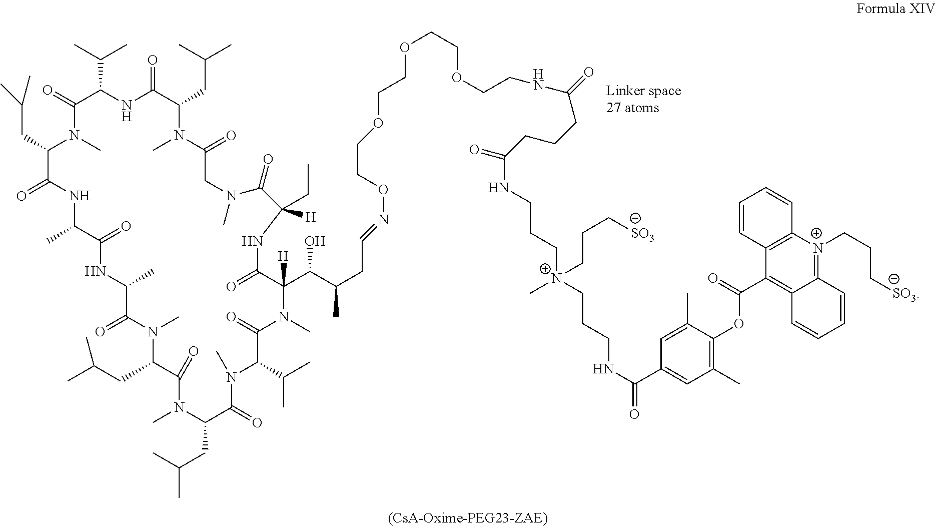

In a particular (but non-limiting) embodiment, the composition is a Cyclosporine A-Acridinium Ester having the structure of Formula XV:

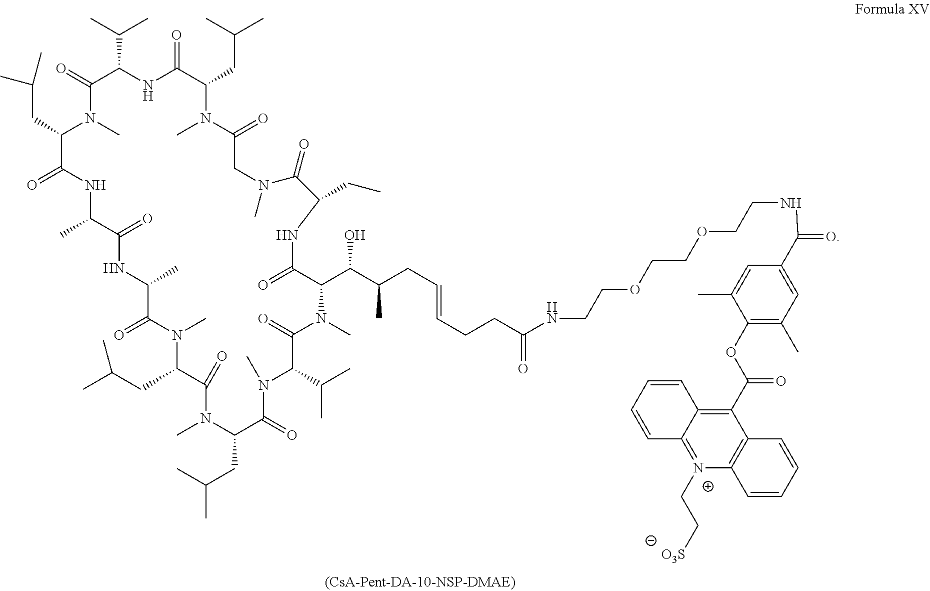

In a particular (but non-limiting) embodiment, the composition is a Cyclosporine A-Acridinium Ester having the structure of Formula XVI:

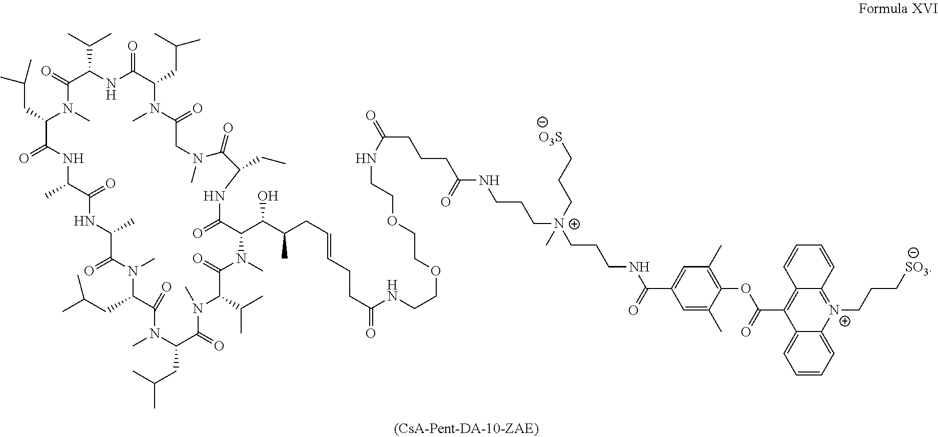

In a particular (but non-limiting) embodiment, the composition is a Cyclosporine A-Acridinium Ester having the structure of Formula XVII:

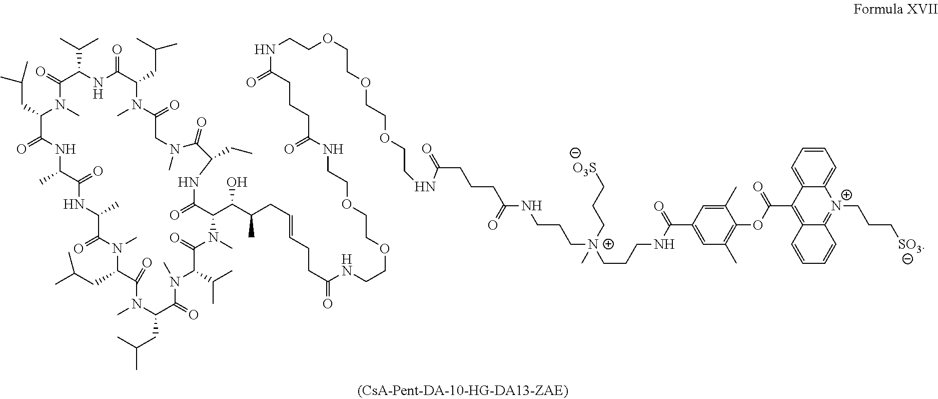

Certain non-limiting embodiments of the present disclosure are directed to an immunoassay kit that contains one or more of any of the CsC-AE/CsA-AE compositions disclosed or otherwise contemplated herein. The selection of the acridinium ester present will depend on the particular assay format to be utilized, and such selection is well within the purview of a person having ordinary skill in the art.

In certain particular (but non-limiting) embodiments, the immunoassay kit includes a first reagent comprising any of the CsC-AE/CsA-AE compositions described or otherwise contemplated herein and a second reagent comprising a solid phase having an antibody directly or indirectly attached thereto; the antibody present in the second reagent specifically binds to the CsC or CsA present in the first reagent.

Any antibodies or fragments thereof known in the art or otherwise contemplated herein may be utilized in accordance with the present disclosure, so long as the antibody/fragment thereof can bind to the CsC or CsA at an epitope that is separate from the position to which the acridinium ester is attached. Antibodies that bind to CsC/CsA are well known in the art and commercially available. For example, but not by way of limitation, CsC/CsA antibodies are commercially available from ThermoFisher Scientific (Waltham, MA); LifeSpan Biosciences (Seattle, WA); MyBioSource (San Diego, CA); Novus Biologicals (Littleton, CO); GeneTex (Irvine, CA); Enzo Life Sciences, Inc. (Farmingdale, NY); Creative Biolabs (Shirley, NY); Santa Cruz Biotechnology, Inc. (Dallas, TX); (Abbexa Ltd (Cambridge, UK); Absolute Antibody (Oxford, UK); Biorbyt (Cambridge, UK); (HyTest Ltd (Turku, Finland); and the like. Therefore, no further description of the antibodies utilized in accordance with the present disclosure is deemed necessary.

The assay reagents present in the kits may be provided in any form that allows them to function in accordance with the present disclosure. For example, but not by way of limitation, each of the reagents may be provided in liquid form and disposed in bulk and/or single aliquot form within the kit. Alternatively, in a particular (but non-limiting) embodiment, one or more of the reagents may be disposed in the kit in the form of a single aliquot lyophilized reagent. The use of dried reagents in microfluidics devices is described in detail in U.S. Pat. No. 9,244,085 (Samproni), the entire contents of which are hereby expressly incorporated herein by reference.

In addition to the assay reagents described in detail herein above, the kits may further contain other reagent(s) for conducting any of the particular assays described or otherwise contemplated herein. For example (but not by way of limitation), the kit may further include at least one pretreatment/releasing agent for releasing the cyclosporine from any endogenous binding proteins present in the biological sample. The nature of these additional reagent(s) will depend upon the particular assay format, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the cross-reactivity and stability of the components/reagents. In addition, the kit may include a microfluidics device in which the components/reagents are disposed.

The relative amounts of the various components/reagents in the kits can vary widely to provide for concentrations of the components/reagents that substantially optimize the reactions that need to occur during the assay methods and further to optimize substantially the sensitivity of an assay. Under appropriate circumstances, one or more of the components/reagents in the kit can be provided as a dry powder, such as a lyophilized powder, and the kit may further include excipient(s) for dissolution of the dried reagents; in this manner, a reagent solution having the appropriate concentrations for performing a method or assay in accordance with the present disclosure can be obtained from these components. Non-limiting examples of other reagents that can be included in the kits include wash solutions, dilution solutions, excipients, interference solutions, positive controls, negative controls, calibration reagents, quality control reagents, and the like. In addition, the kit can further include a set of written instructions explaining how to use the kit. A kit of this nature can be used in any of the methods described or otherwise contemplated herein.

Certain non-limiting embodiments of the present disclosure are directed to a method of producing any of the CsC-AE/CsA-AE compositions disclosed or otherwise contemplated herein. The method includes attaching a linker to the CsC/CsA and then attaching the acridinium ester to the linker.

Certain non-limiting embodiments of the present disclosure are directed to a method of detecting CsC/CsA in a sample utilizing any of the CsC-AE/CsA-AE compositions disclosed or otherwise contemplated herein. In the method, a sample suspected of containing CsC/CsA is combined, either simultaneously or wholly or partially sequentially, with one or more of any of the CS-AE compositions disclosed or otherwise contemplated herein and one or more of the CsC/CsA-antibodies associated with a solid phase as disclosed or otherwise contemplated herein to form a mixture, and the mixture is incubated under conditions that allow for binding of the antibody to the CsC/CsA present in the sample or to the CsC-AE/CsA-AE, thereby forming a complex of Cs/antibody and/or a complex of Cs-AE/antibody. Then the complex of Cs-AE/antibody is detected, and an amount of CsC/CsA present in the sample is determined based upon a reduction in an amount of Cs-AE/antibody complex formed when compared to a negative control (i.e., an amount of Cs-AE/antibody complex formed in the absence of sample). A concentration of CsC/CsA present in the sample can then be determined based upon the amount of reduction.

In particular (but non-limiting) embodiments of the method, a sample is combined, either simultaneously or wholly or partially sequentially, with the first and second reagents of the immunoassay kit described in detail herein above (i.e., a first reagent comprising the CsC/CsA-AE composition and a second reagent comprising a solid phase having an antibody or fragment thereof that specifically binds to CsC/CsA directly or indirectly attached thereto) to form a mixture. The mixture is then incubated under conditions that will allow for binding of the second reagent to any target analyte (CsC or CsA) present in the sample, or to the first reagent, thereby forming a complex of Cs/antibody and/or a complex of Cs-AE/antibody. The complex of first reagent-second reagent (Cs-AE/antibody) is then detected by any method known in the art, and an amount of target analyte (CsC or CsA) present in the sample is determined based upon a reduction in an amount of first reagent-second reagent complex formed when compared to a negative control (i.e., an amount of first reagent-second reagent complex formed in the absence of sample). A concentration of target analyte (CsC or CsA) present in the sample is then determined based upon the amount of reduction.

Any sample for which an assay for the presence of a cyclosporine target analyte (i.e., CsC or CsA) is desired can be utilized as the sample in accordance with the methods of the present disclosure. Non-limiting examples of samples include a biological sample such as, but not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, and combinations thereof. Particular non-limiting examples include lysed whole blood cells and lysed red blood cells.

As mentioned above, the various components of the method are provided in combination (either simultaneously or sequentially). When the various components of the method are added sequentially, the order of addition of the components may be varied; a person having ordinary skill in the art can determine the particular desired order of addition of the different components to the assay. The simplest order of addition, of course, is to add all the materials simultaneously and determine the signals produced therefrom. Alternatively, each of the components, or groups of components, can be combined sequentially. In certain embodiments, an incubation step may be involved subsequent to one or more additions. For example (but not by way of limitation), it may be desirable to combine and incubate the sample with the antibody-solid phase prior to the addition of the Cs-AE composition.

The conditions under which the mixture(s) is incubated can vary widely, so long as the antibody associates with the Cs or Cs-AE to form a complex under such conditions. Immunoassays based on a sandwich assay format are widely performed, and immunoassay conditions are well known in the art; thus, selection of appropriate assay conditions is well within the purview of a person having ordinary skill in the art, and thus no further description thereof is deemed necessary.

The particular detection method utilized can vary widely, so long as the complex can be detected under such methods. Detection of complexes formed in a sandwich assay format are widely performed, and the detection procedures are well known in the art; thus, selection of appropriate detection methods is well within the purview of a person having ordinary skill in the art, and thus no further description thereof is deemed necessary.

The method may further include one or more additional steps to increase the accuracy and/or precision of the assay. For example (but not by way of limitation), the method may further include one or more pretreatment/releasing steps for releasing the cyclosporine from any endogenous binding proteins present in the biological sample before combining with the immobilized antibody. Another non-limiting example of an additional step that may be utilized in accordance with the present disclosure include one or more wash steps for removing unbound (or non-specifically bound) reagent from the reaction prior to detection of complex formation.

Certain additional non-limiting embodiments of the present disclosure are directed to a microfluidics device that includes the components of any of the immunoassay kits described herein above. In particular, certain non-limiting embodiments include a microfluidics device for detecting target analyte (CsC or CsA) in a sample. The microfluidics device comprises (i) an inlet channel through which a sample is applied; and (ii) at least a first compartment capable of being in fluidic communication with the inlet channel. The compartment(s) of (ii) contains the first and second reagents of the immunoassay kit described in detail herein above.

In certain non-limiting embodiments, the first and second reagents (as well as any additional elements, as described herein above) of (ii) are present in the same compartment. In alternative non-limiting embodiments, the first and second reagents (as well as any additional elements, as described herein above) are split between two or more compartments.

The device may be provided with any arrangement of the compartments and distribution of the various components therebetween that allows the device to function in accordance with the present disclosure.

Any of the compartments of the microfluidics device may be sealed to maintain reagent(s) disposed therein in a substantially air tight environment until use thereof; for example, compartments containing lyophilized reagent(s) may be sealed to prevent any unintentional reconstitution of the reagent. The inlet channel and a compartment, as well as two compartments, may be described as being “capable of being in fluidic communication” with one another; this phrase indicates that each of the compartment(s) may still be sealed, but that the two compartments are capable of having fluid flow therebetween upon puncture of a seal formed therein or therebetween.

The microfluidics devices of the present disclosure may be provided with any other desired features known in the art or otherwise contemplated herein. For example, but not by way of limitation, the microfluidics devices of the present disclosure may further include a read chamber; the read chamber may be any of the compartments containing one or more of the reagents described herein above, or the read chamber may be in fluidic communication with said compartment(s) containing one or more reagents. The microfluidics device may further include one or more additional compartments containing other solutions, such as (but not limited to) wash solutions, dilution solutions, excipients, interference solutions, positive controls, negative controls, quality controls, and the like. These additional compartment(s) may be in fluidic communication with one or more of the other compartments. For example, the microfluidics device may further include one or more compartments containing a wash solution, and these compartment(s) may be capable of being in fluidic communication with any other compartment(s) of the device. In another example, the microfluidics device may further include one or more compartments containing an excipient for dissolution of one or more dried reagents, and the compartment(s) may be capable of being in fluidic communication with any other compartment(s) of the device. In yet a further example, the microfluidics device may include one or more compartments containing a dilution solution, and the compartment(s) may be capable of being in fluidic communication with any other compartment(s) of the device.

### Example

An Example is provided hereinbelow. However, the present disclosure is to be understood to not be limited in its application to the specific experimentation, results, and laboratory procedures disclosed herein. Rather, the Example is simply provided as one of various embodiments and is meant to be exemplary, not exhaustive.

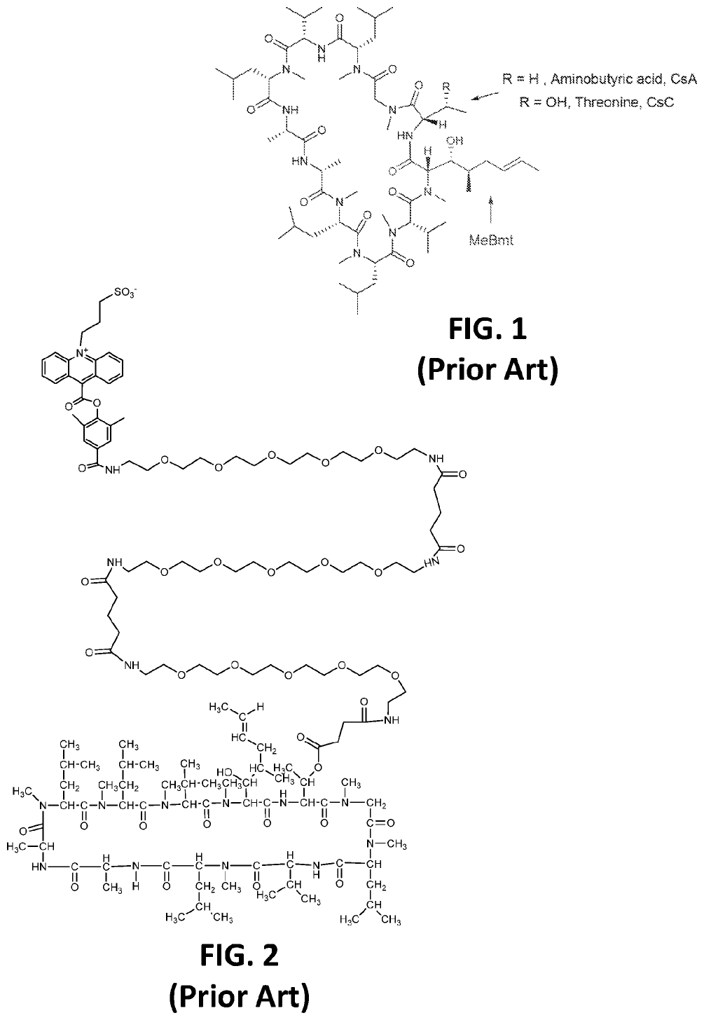

Cyclosporine A AIP assays are currently used for in vitro diagnostic use in the quantitative determination of cyclosporine in human whole blood (EDTA) using the ADVIA® CENTAUR® and ATELLICA® IM Analyzers (Siemens Healthcare Diagnostics Inc., Tarrytown, NY). This assay is intended for use as an aid in the management of cyclosporine therapy in kidney, heart, and liver transplant patients. The chemical structures of Cyclosporine A (CsA) and Cyclosporine C (CsC) are shown in FIG. 1. The only structural difference between CsA and CsC is the additional hydroxy group on the CsC molecule (FIG. 1). The original ADVIA® CENTAUR® Cyclosporine (CsA) Assay was commercialized in 2008, and this original immunoassay employed a complex CsC-Acridinium Ester (CsC-AE) conjugate (FIG. 2) for the detection of CsA. However, the vendor discontinued the raw material CsC-succinate-NHS utilized to produce this conjugate. Therefore, there was a need for new AE tracers designed for the CsA II assay.

In addition, a novel Cs assay has been designed that addresses the European REACH (Registration, Evaluation, Authorisation, and Restriction of Chemicals) requirement for Triton reduction, as well as the US FDA's ambient temperature effort requirement (ATE) and biotin interference mitigation. The novel Cs assays of the present disclosure utilize new tracers, and the design process emphasized the identification of new tracers that have simplified chemical structures and can be produced with convenient synthesis processes.

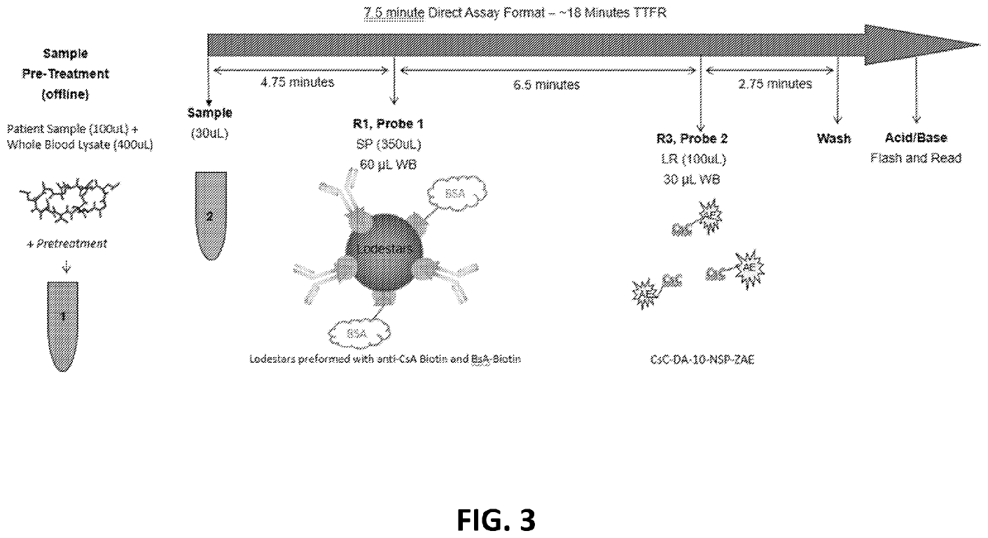

Based on this need, a series of novel and simple Cyclosporine (A and C)-Acridinium Esters (CsA-AEs and CsC-AEs) were designed and synthesized for investigation in an ADVIA® CENTAUR® CSA II assay (Siemens Healthcare Diagnostics Inc., Tarrytown, NY), based upon acridinium ester (AE) technology (Natrajan et al., Ann Biochem (2010) 406:204; and Natrajan et al., Org Biomol Chem (2011) 9:5092)). These CsA-AEs and CsC-AEs were evaluated on the ADVIA® CENTAUR® system (Siemens Healthcare Diagnostics Inc., Tarrytown, NY) using a competitive assay format (FIG. 3). One important key tracer, CsC-DA-10-NSP-ZAE (Formula IX; FIG. 7), demonstrated excellent performance relative to assay requirements (ATE, sensitivity, and accuracy) and was chosen as the primary candidate tracer for the CsA II immunoassay.

Reagent Design: The ADVIA® CENTAUR®/ATELLICA® CSA II reagents included one solid phase and one lite reagent. The solid phase reagent contained magnetic particles labeled with biotinylated monoclonal anti-CsA antibody. The lite reagent contained the CsC-AE tracer. In the absence of CsA in the test sample, the lite reagent was bound by the antibody located on the surface of the solid phase (FIG. 3). The bound AE remaining in the reaction vessel following magnetic separation and washing of the particles gives off light when activated by the addition of acid and base. The addition of CsA from a patient sample to the reaction disrupts the binding of the Cs-AE conjugate to the solid phase, resulting in a decrease in signal generation. This decrease in signal is a direct function of the amount of CsA in the sample when measured against a calibration curve.

Given the above assay design, both solid phase reagents and novel CsC-AE tracers were developed and evaluated. Preparation of biotinylated monoclonal antibodies and pre-binding of these antibodies to the surface of commercial streptavidin-coated particles is a well-known and straight forward process. Hence, the remainder of this Example focuses on the syntheses of new CsC-AE and CsA-AE tracers and their performances in the prototype ADVIA Centaur CsA II assay.

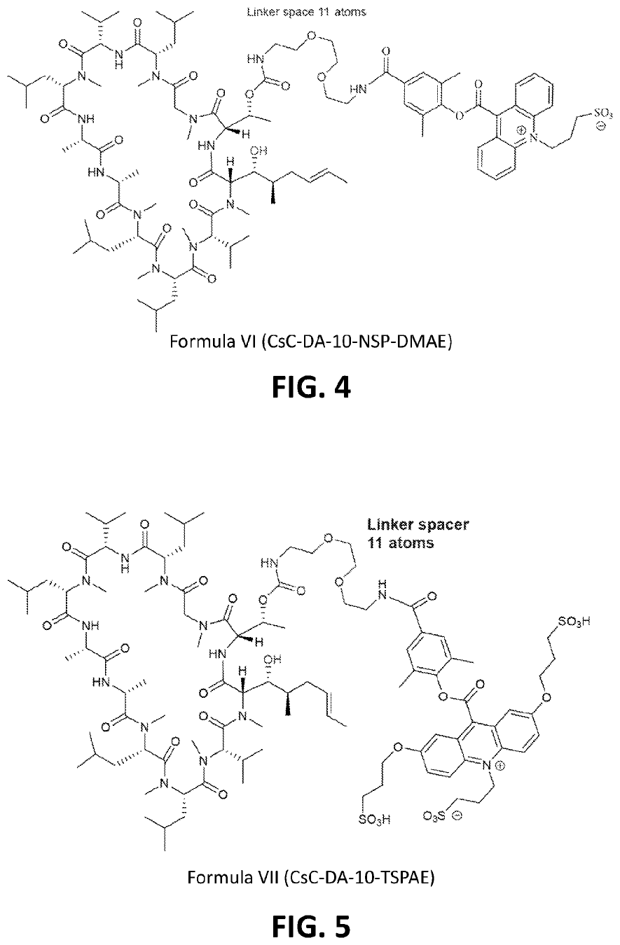

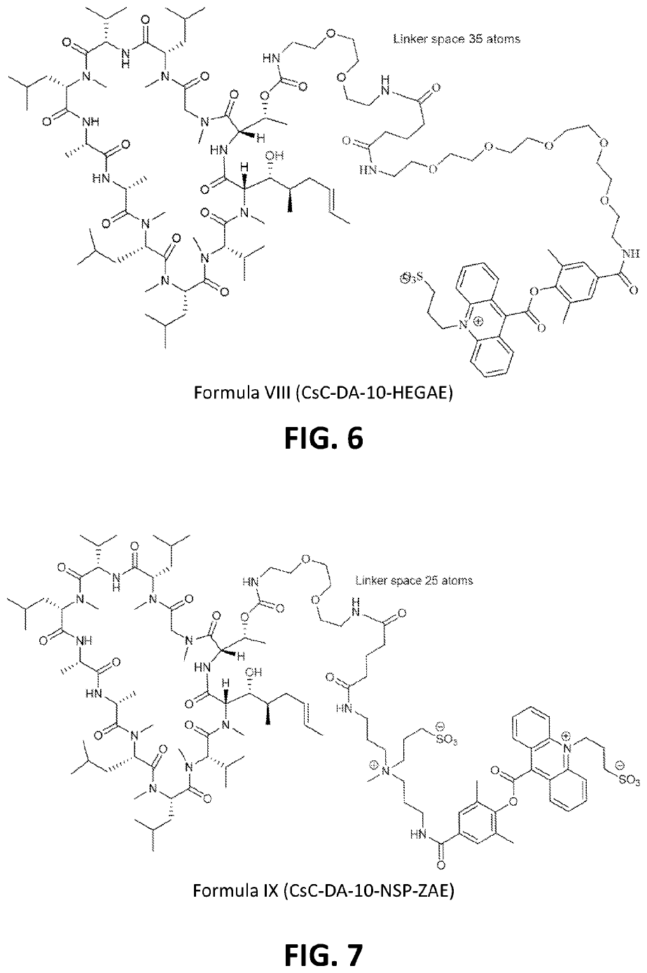

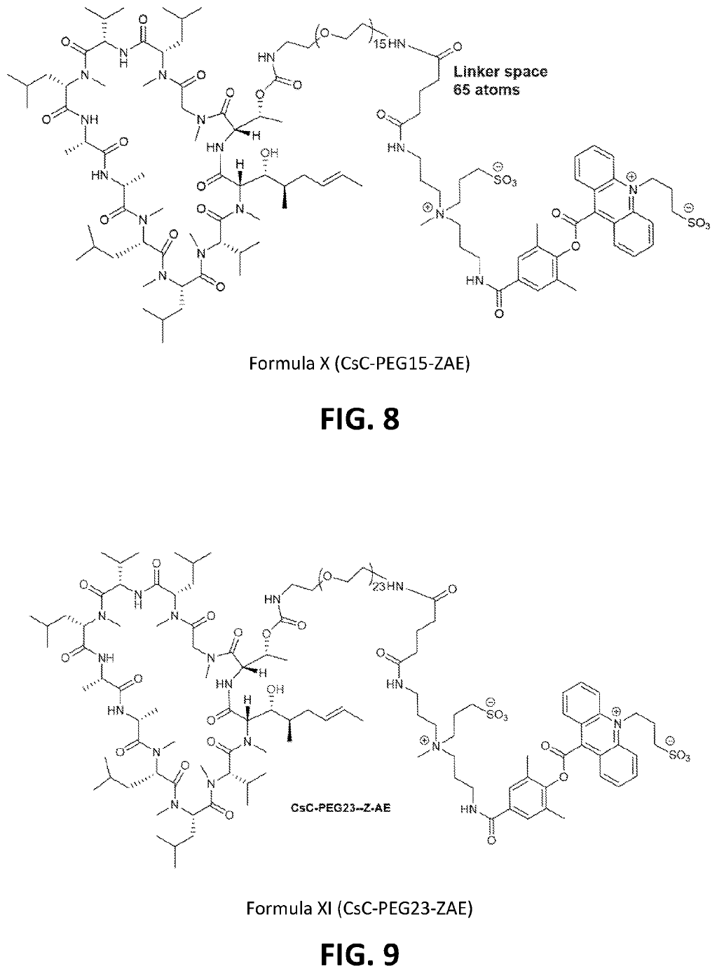

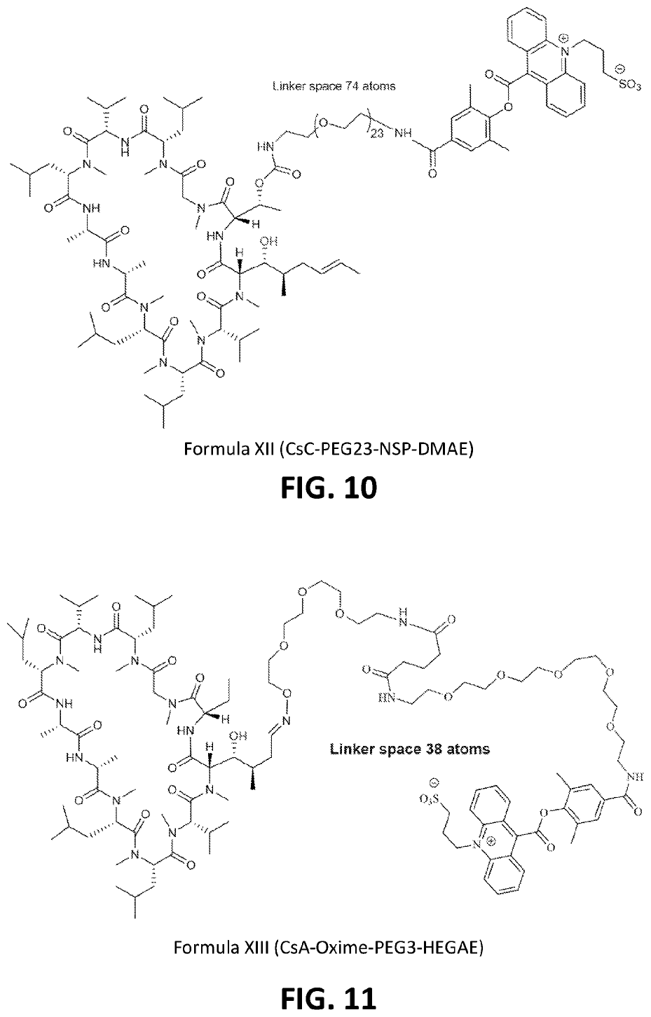

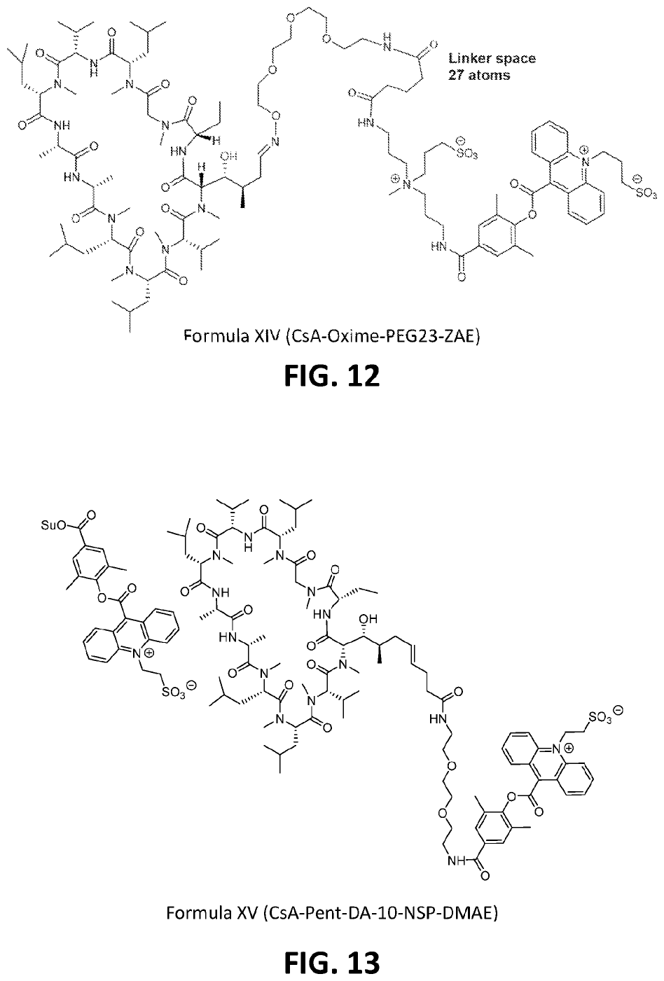

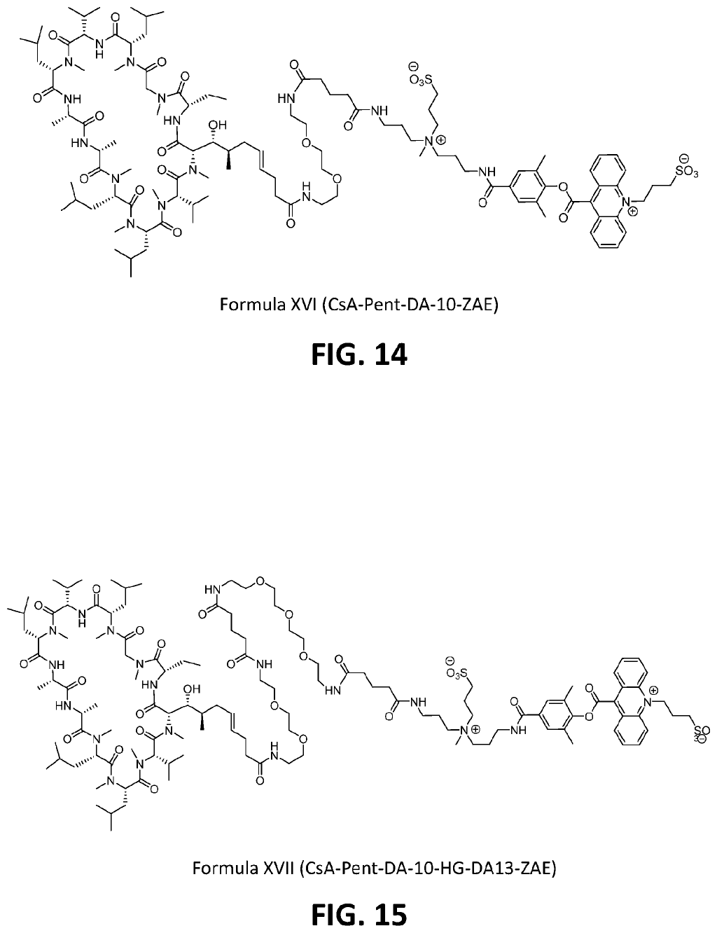

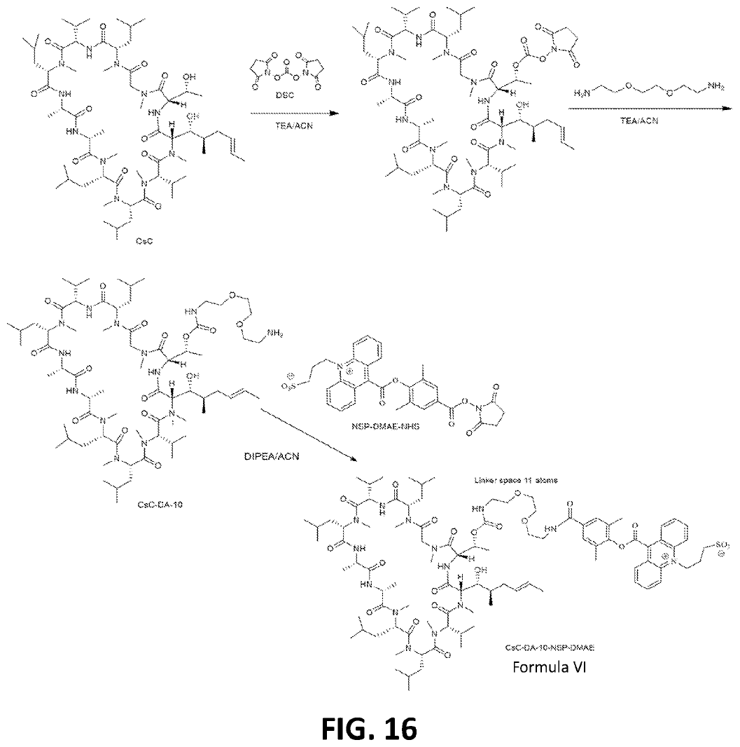

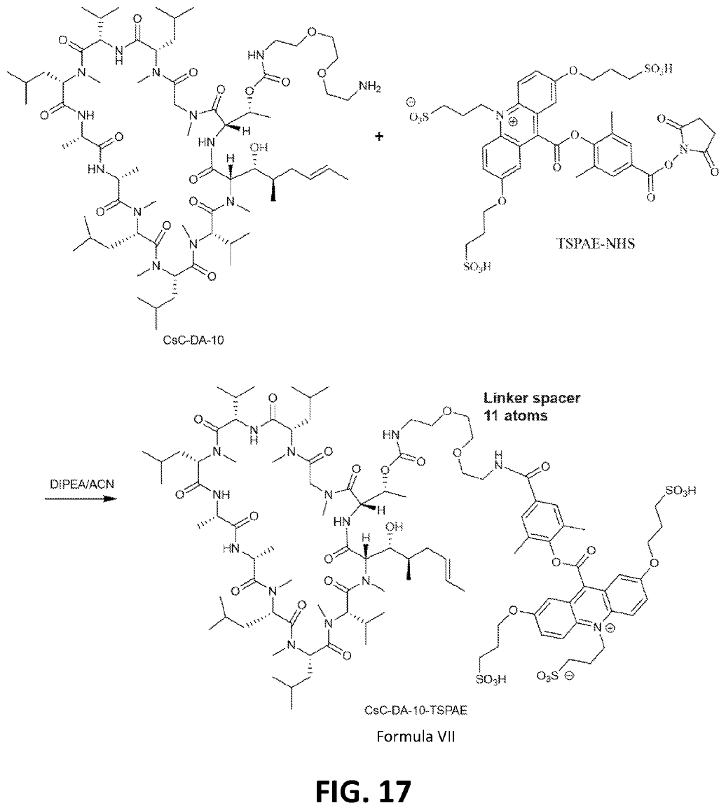

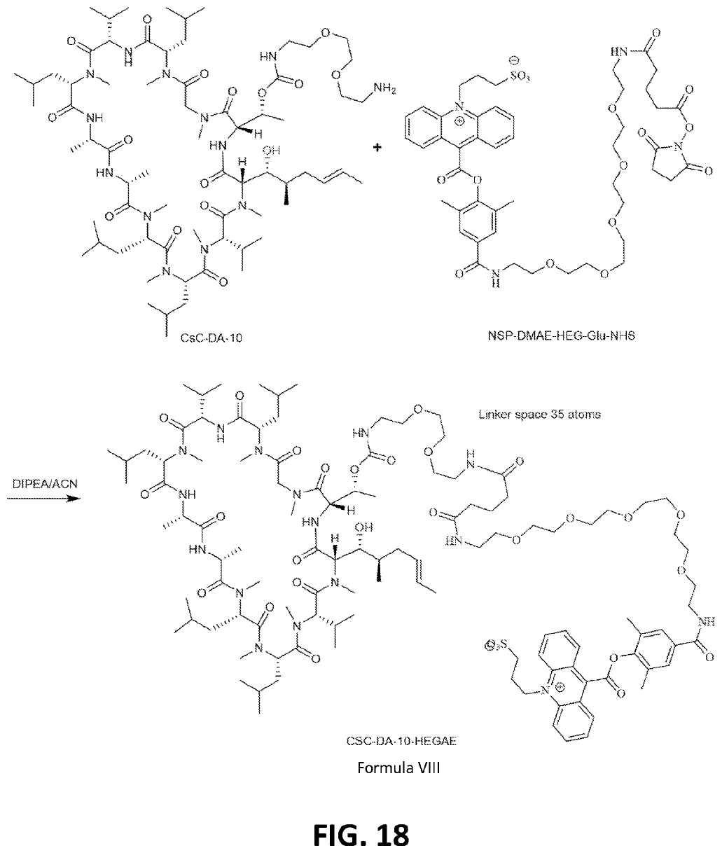

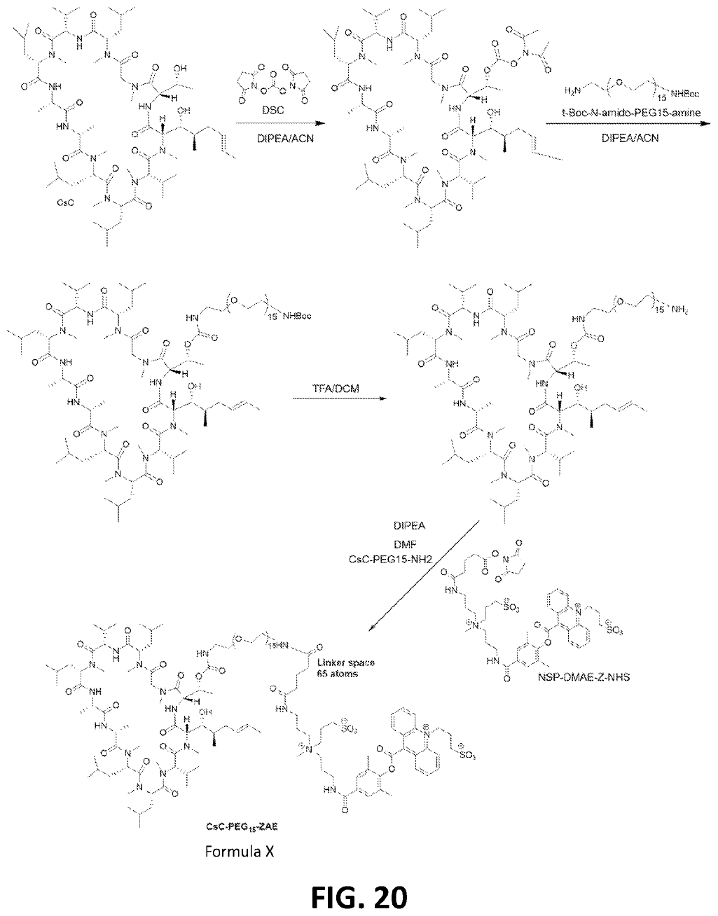

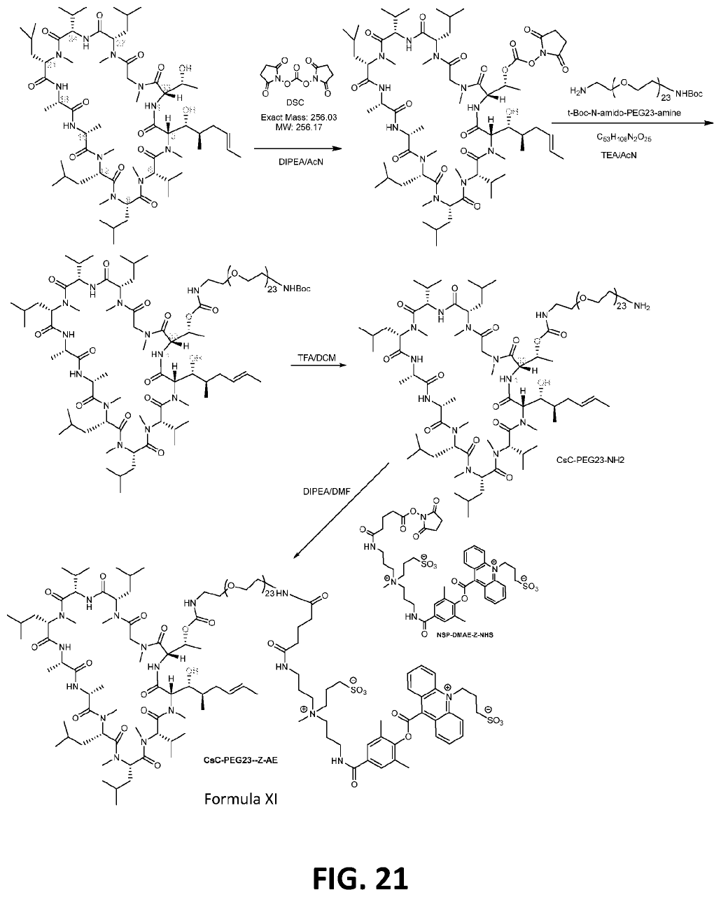

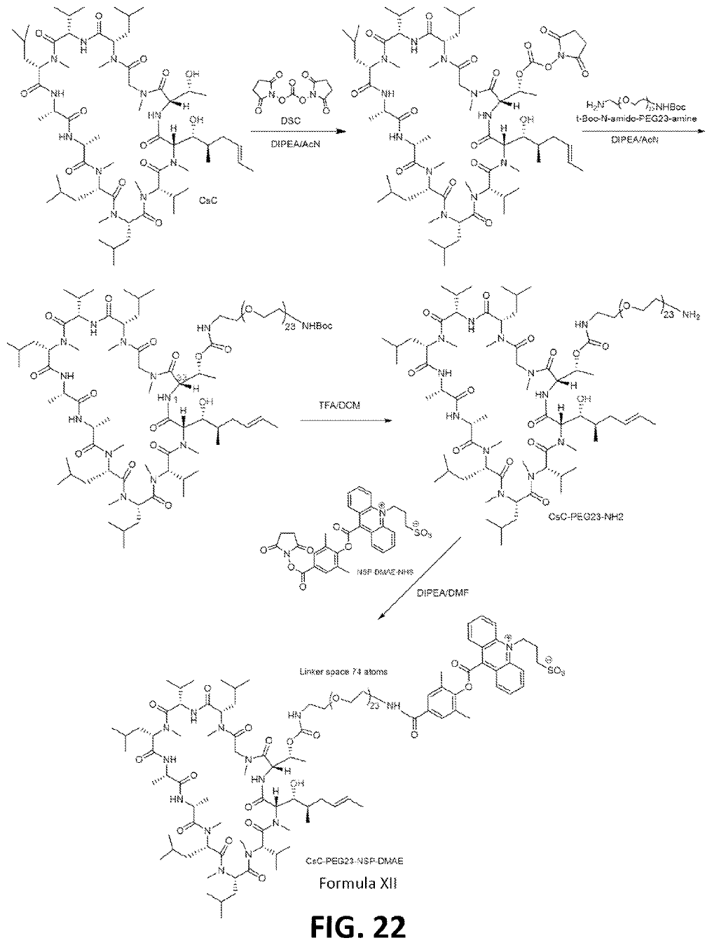

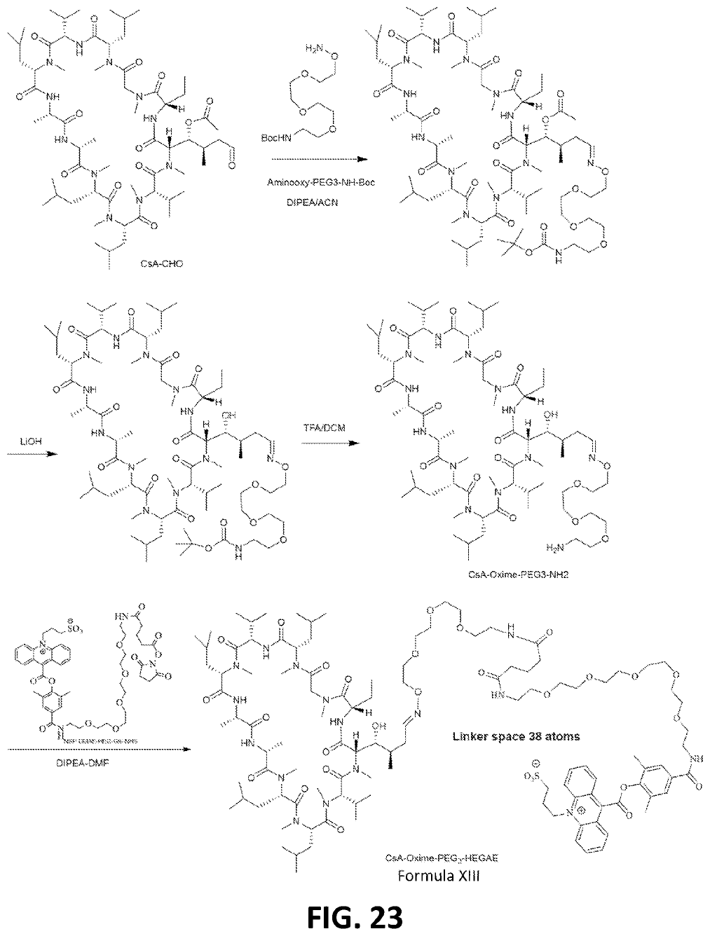

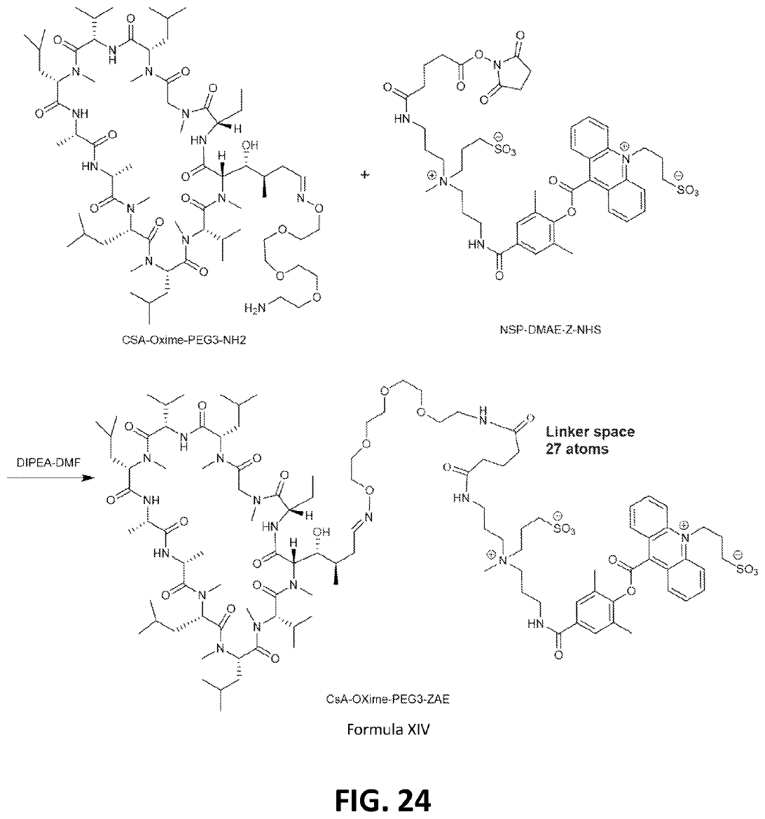

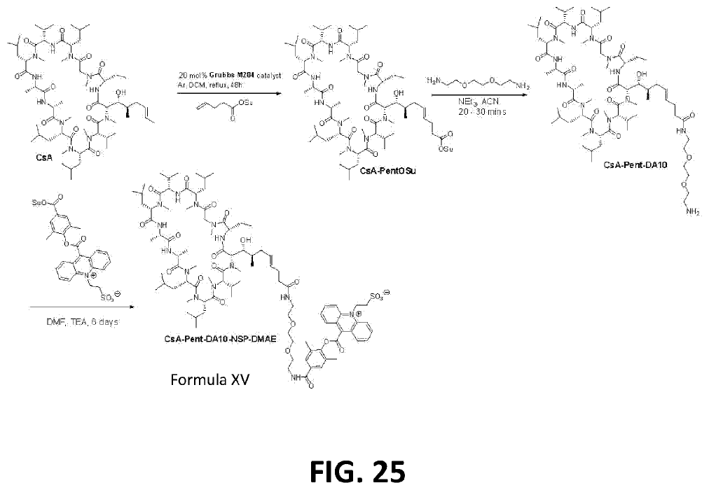

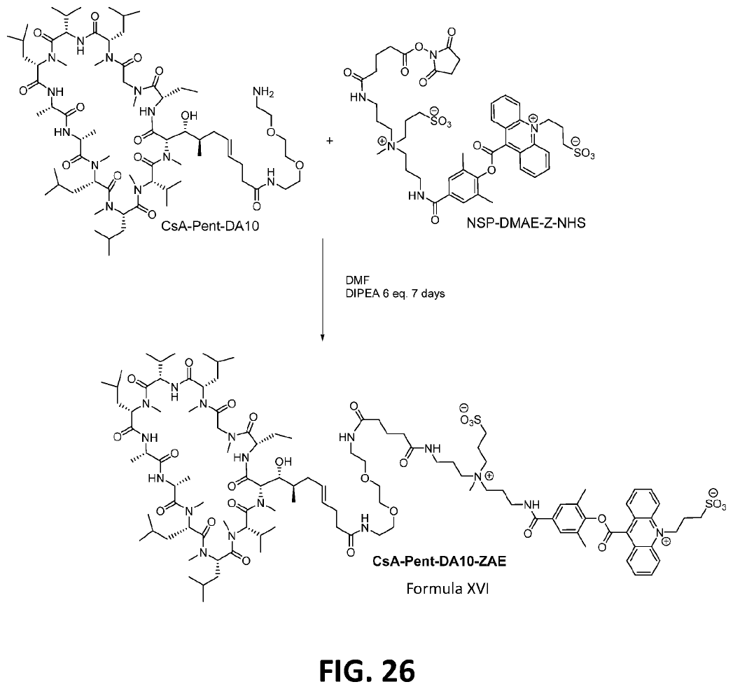

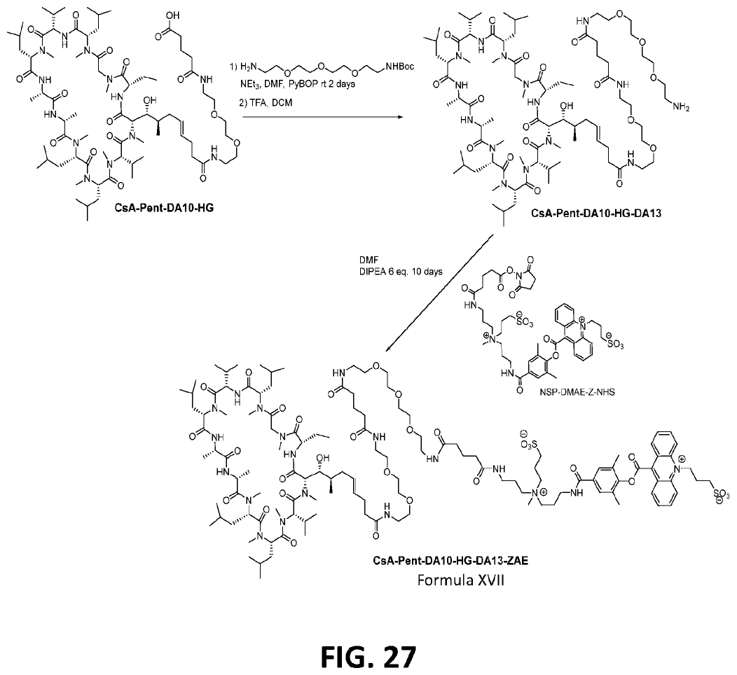

A hydroxy group (FIG. 1) from the CsC molecule was selected for NSP-DMAE conjugation in initial assay studies. Early data in this Example indicated that linkage to this hydroxy of CsC-DA-10-NSP-DMAE yielded promising assay curve shape. Given this initial performance, conjugates were also prepared with HEGAE, TSPAE, and ZAE at the same linkage position, to determine if the differing AE structures would be of any additional benefit to the assay. Also, the terminal double and aldehyde functions of CsA (FIG. 1) were used as a basis to introduce NSP-DMAE and ZAE. The chemical structures of CsA-AEs and CsC-AEs tracers are shown in FIGS. 4-15, and the syntheses of these CsA-AEs and CsC-AEs tracers are presented in FIGS. 16-27.

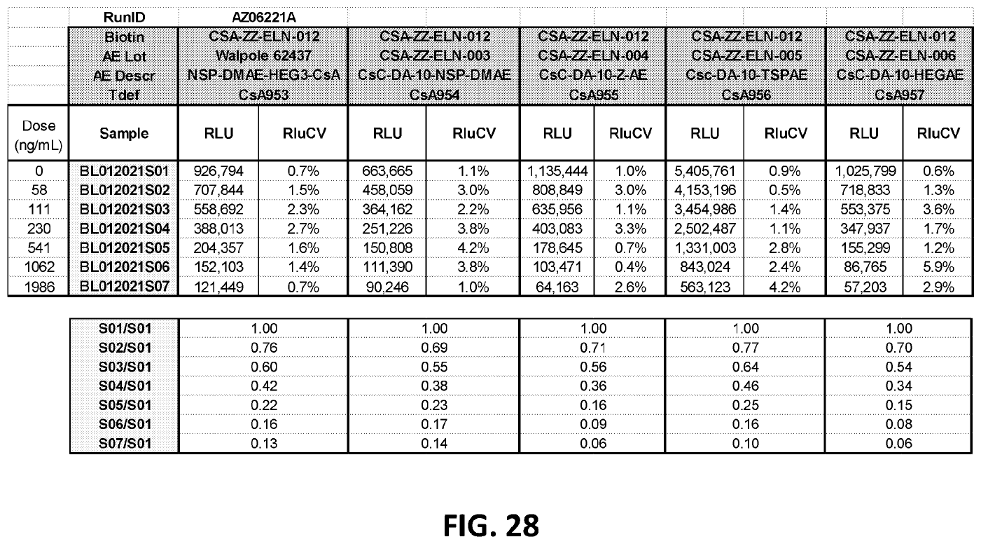

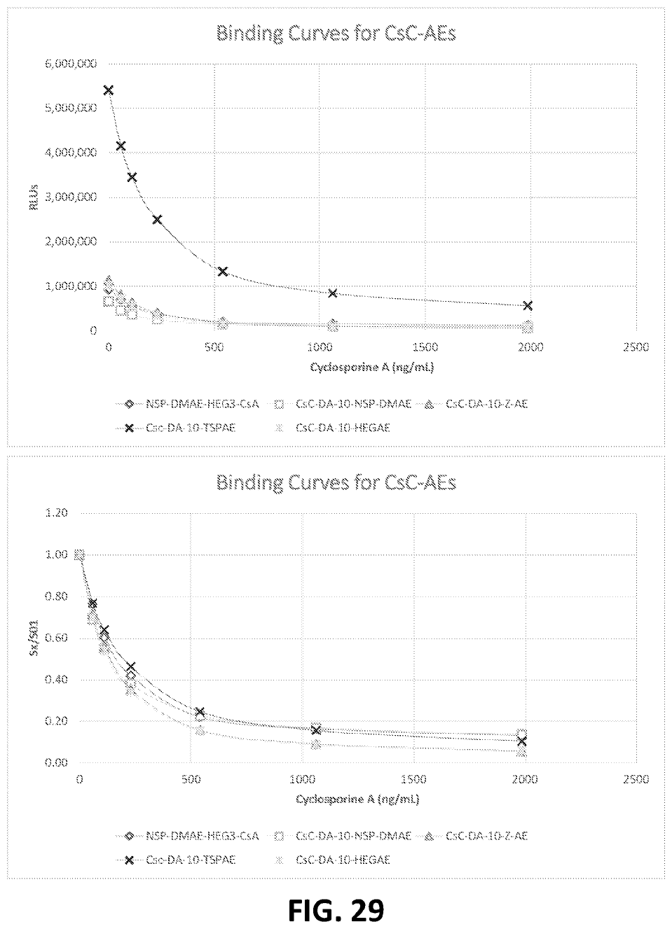

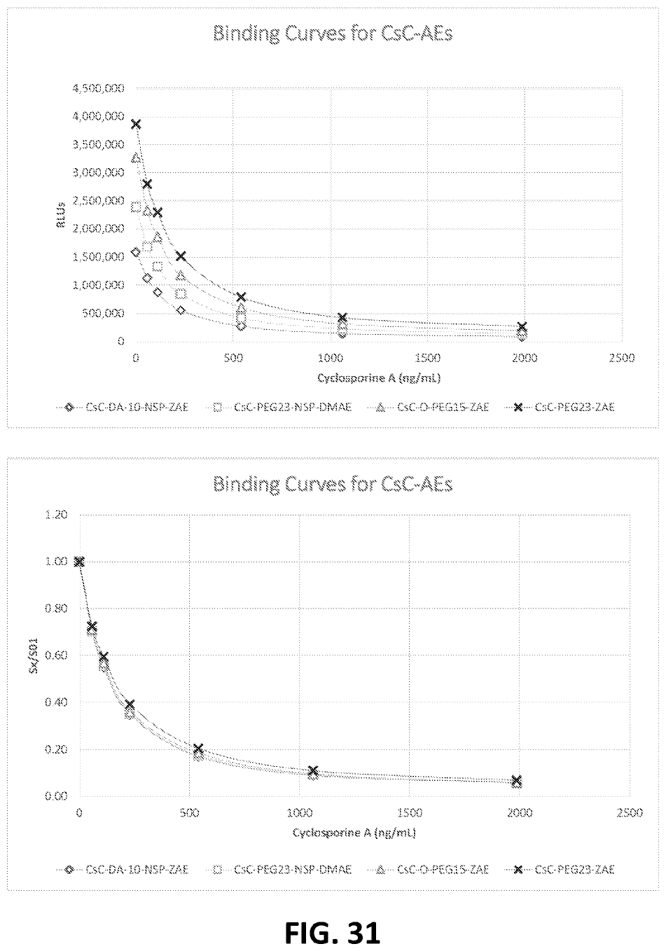

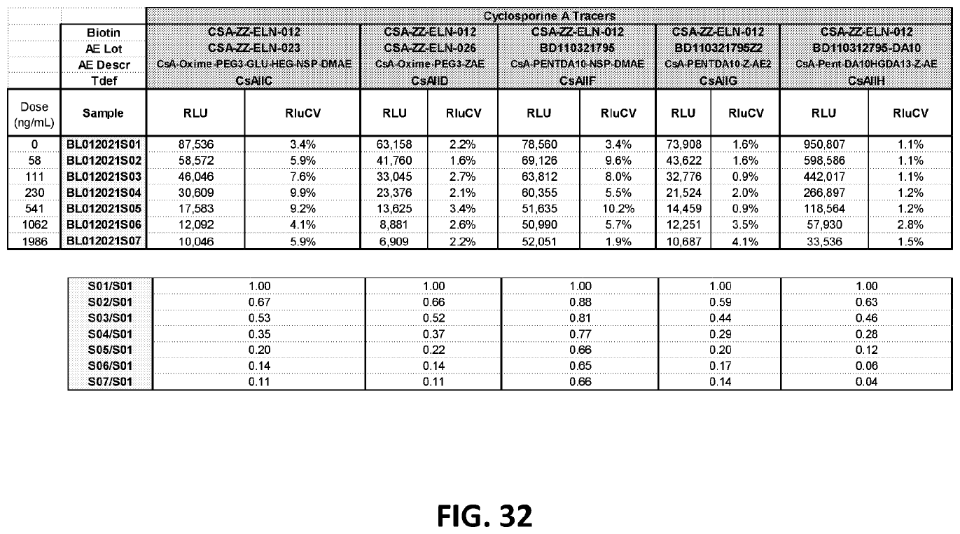

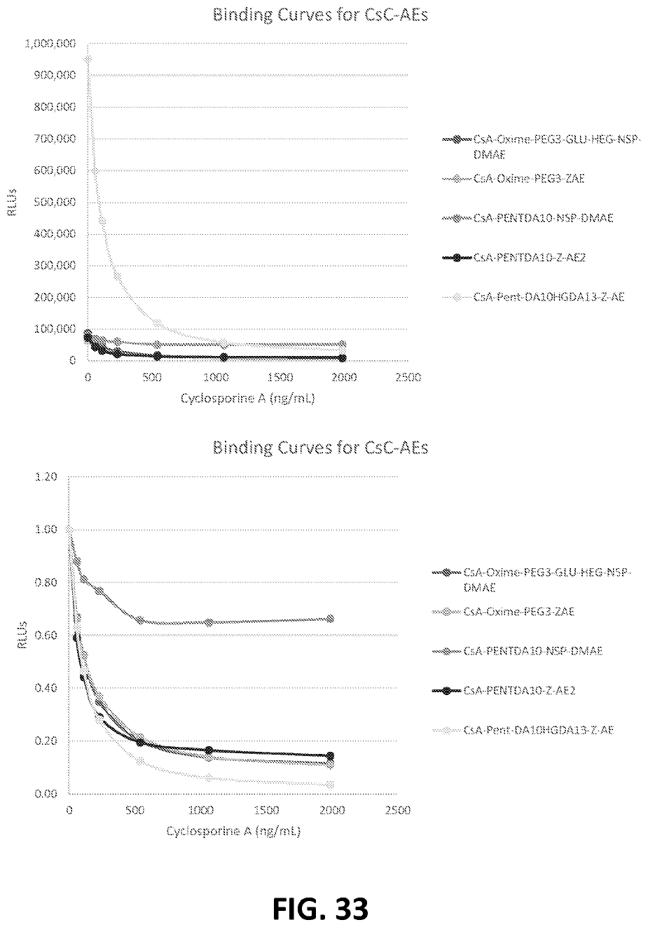

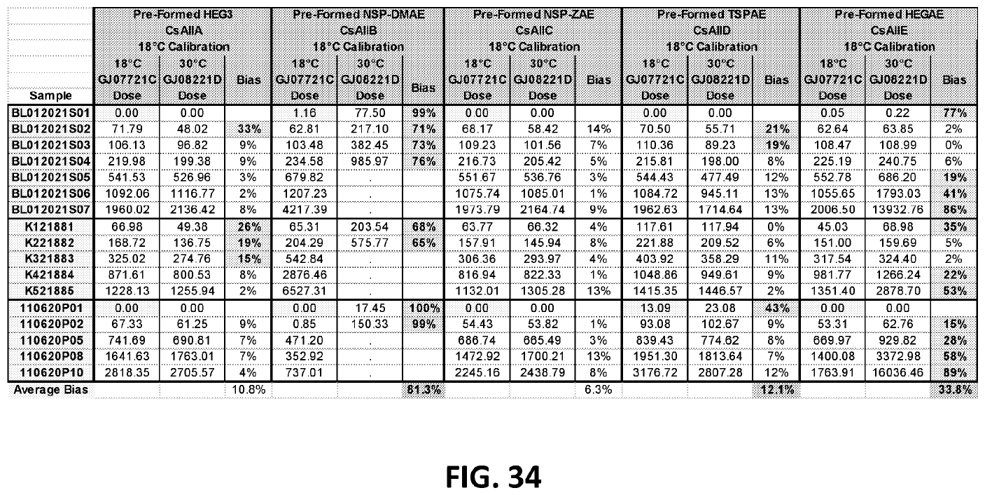

Cyclosporine Immunoassay Performance: Evaluations of the CsA-AEs and CsC-AEs tracers were carried out using the ADVIA® CENTAUR® family of immunoassay analyzers, available from Siemens Healthcare Diagnostics Inc., Tarrytown, NY. The screen for binding of the CsA-AEs and CsC-AEs tracers (FIGS. 4-15) to available monoclonal antibodies was determined using a prototype ADVIA® CENTAUR® Cs immunoassay (FIGS. 28-33). An ambient temperature effort (ATE) study using different CsC tracers is also shown in FIGS. 34-36.

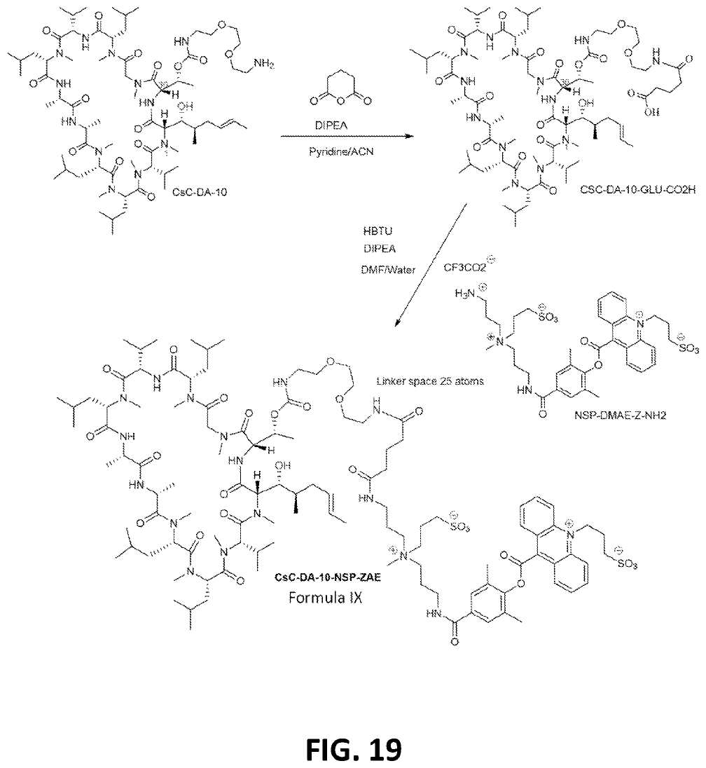

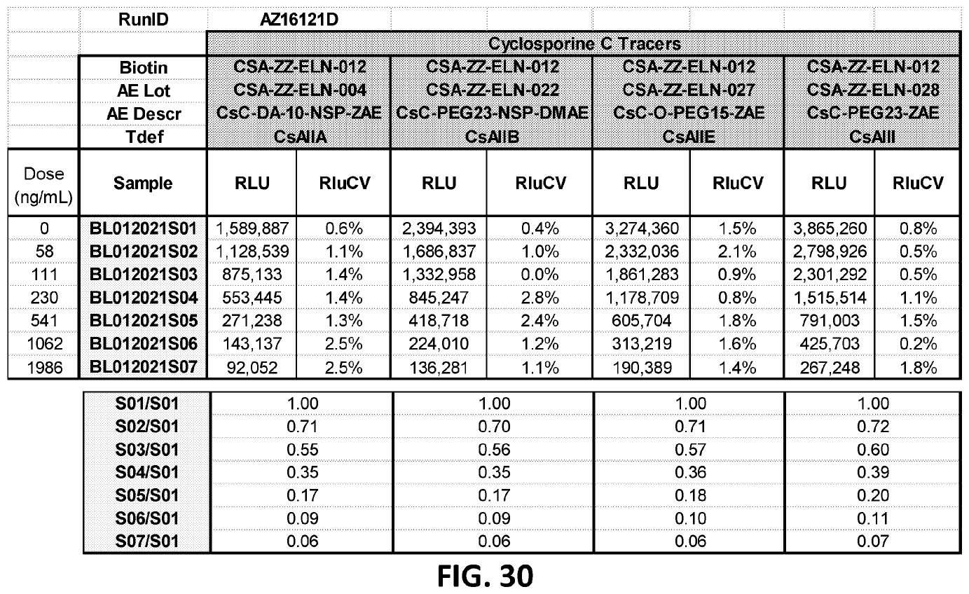

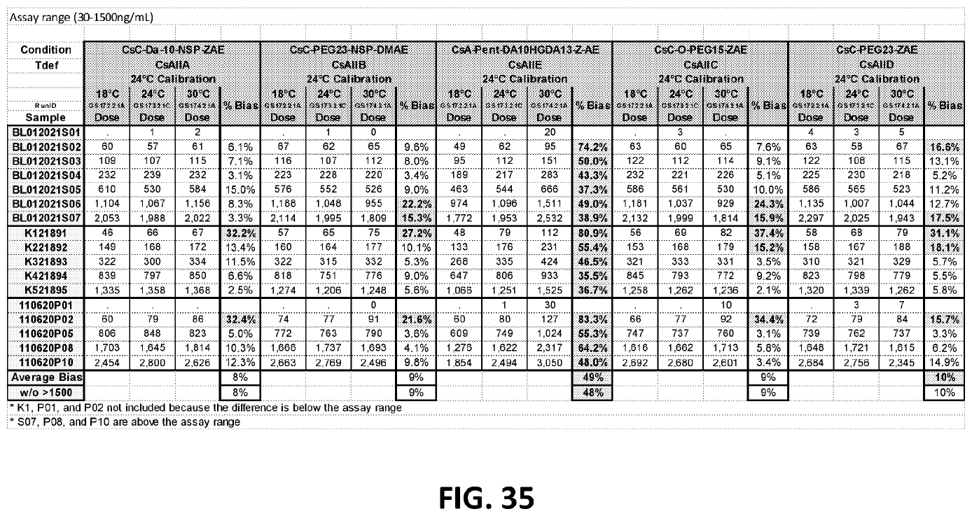

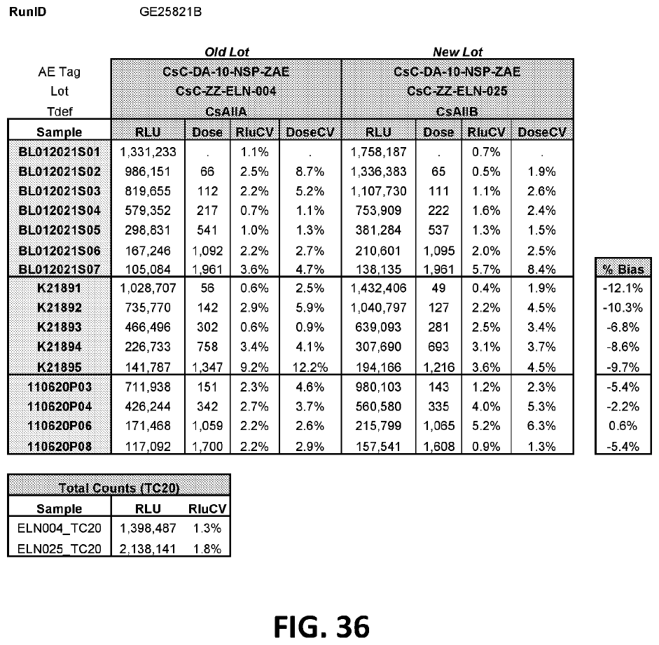

Conclusion: A series of new CsA-AEs and CsC-AEs tracers have been successfully designed, synthesized, and evaluated in a prototype CsA immunoassay (FIGS. 4-15; FIGS. 16-27; and FIGS. 28, 30, 32, and 34-36; respectively). A novel tracer was selected as an excellent candidate for meeting the FDA's ambient temperature effort requirement and overall assay performance/assay specification (FIGS. 30 and 35). This tracer, CsC-DA-10-NSP-ZAE (Formula IX; FIG. 7), has a novel simplified chemical structure which can be conveniently synthesized (FIG. 19) in comparison to that of the currently used complex tracer CsA-HEG3-NSP-DMAE (FIG. 2). This saves production time and cost and overcomes the issue of discontinued raw material for the current tracer. The new tracers developed in accordance with the present disclosure are thus critical components for the development of the new Cs immunoassay.

### NON-LIMITING ILLUSTRATIVE EMBODIMENTS OF THE INVENTIVE CONCEPTS

Illustrative embodiment 1. A composition, comprising: a cyclosporine C and an acridinium ester linked via a spacer and having the structure of Formula I:

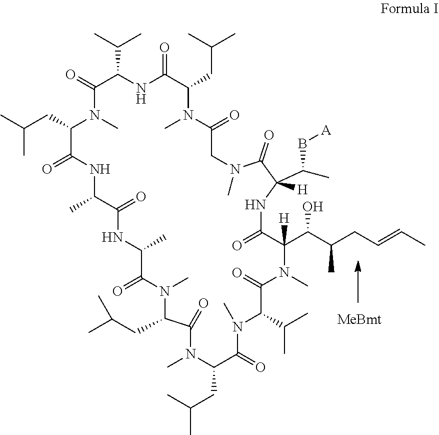

wherein: “A” comprises an acridinium ester; and “B” is a spacer having from about 5 atoms to about 100 atoms, each selected from the group consisting of C, H, O, N, S, and P atoms.

Illustrative embodiment 2. The composition of illustrative embodiment 1, wherein “A” of Formula I is an acridinium ester having the structure of Formula III:

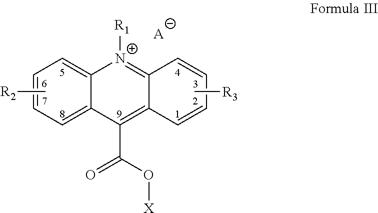

wherein:
“R₁” is selected from the group consisting of: an alkyl, alkenyl, alkynyl, or aralkyl group of 1 to 35 carbon atoms and 0 to 20 heteroatoms; a sulfopropyl or sulfobutyl group; and a group —R^{a}—Z, where R^{a} is a divalent radical selected from alkyl, alkenyl, alkynyl, aryl, or aralkyl group of 1 to 35 carbon atoms and 0 to 20 heteroatoms;
“R₂” is placed at one or more of positions C1 to C4, and each “R₂” is independently selected from the group consisting of hydrogen, alkyl, OR, OH, SR, SH, NH₂, and NR′R″, wherein R, R′, and R″ are each independently selected from the group consisting of an alkyl, alkenyl, alkynyl, aryl, and aralkyl group, wherein each group contains 0 to 20 heteroatoms;
“R₃” is placed at one or more of positions C5 to C8, and each “R₃” is independently selected from the group consisting of hydrogen, alkyl, OR, OH, SR, SH, NH₂, and NR′R″, wherein R, R′, and R″ are each independently selected from the group consisting of an alkyl, alkenyl, alkynyl, aryl, and aralkyl group, wherein each group contains 0 to 20 heteroatoms;
“X” is a group selected from a halogenated or unhalogenated, branched or straight-chained alkyl group; a substituted or unsubstituted aryl group; and a heterocyclic ring group; wherein the “X” group comprises 0 to 20 heteroatoms, and wherein the “X” group further comprises a functional group that links to the spacer “B” of Formula I; and
“A” is a counter ion selected from the group consisting of CH₃SO₃⁻, FSO₃⁻, CF₃SO₃⁻, C₄F₉SO₃⁻, CH₃C₆H₄SO₃⁻, a halide, CF₃COO⁻, CH₃COO⁻, and NO₃⁻.

Illustrative embodiment 3. The composition of illustrative embodiment 2, wherein “A” of Formula I is an acridinium ester having the structure of Formula IV:

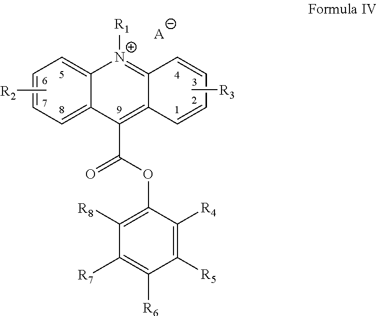

wherein: each of “R₄” and “R₈” is independently selected from hydrogen or an alkyl, alkenyl, alkynyl, alkoxyl (—OR), alkylthiol (—SR), or substituted amino group; each of “R₅,” “R₆,” and “R₇” is independently selected from hydrogen or an alkyl, alkenyl, alkynyl, aryl, or aralkyl group, wherein each group contains 0 to 20 heteroatoms; and one of “R₅,” “R₆,” and “R₇” further comprises a functional group that links to the spacer “B” of Formula I.

Illustrative embodiment 4. The composition of illustrative embodiment 3, wherein “A” of Formula I is a dimethylphenyl acridinium ester having the structure of Formula V:

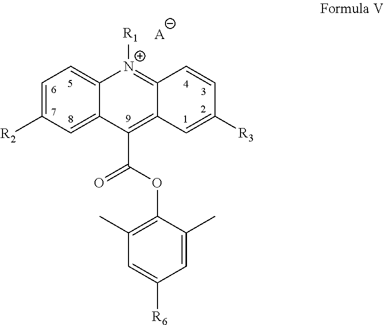

wherein: “R₁” is a methyl or a sulfopropyl group; each of “R₂” and “R₃” is independently selected from hydrogen or a methoxy, sulfopropyloxyl, or poly(ethylene)glycoloxy group; and “R₆” is an amide group (CONH—) connecting to the spacer “B” of Formula I.

Illustrative embodiment 5. The composition of illustrative embodiment 4, wherein R₁ is a sulfopropyl group, and wherein each of R₂ and R₃ is a hydrogen or a sulfopropyloxyl group.

Illustrative embodiment 6. The composition of any one of illustrative embodiments 1-5, further defined as a Cyclosporine C-Acridinium Ester having the structure of Formula VI:

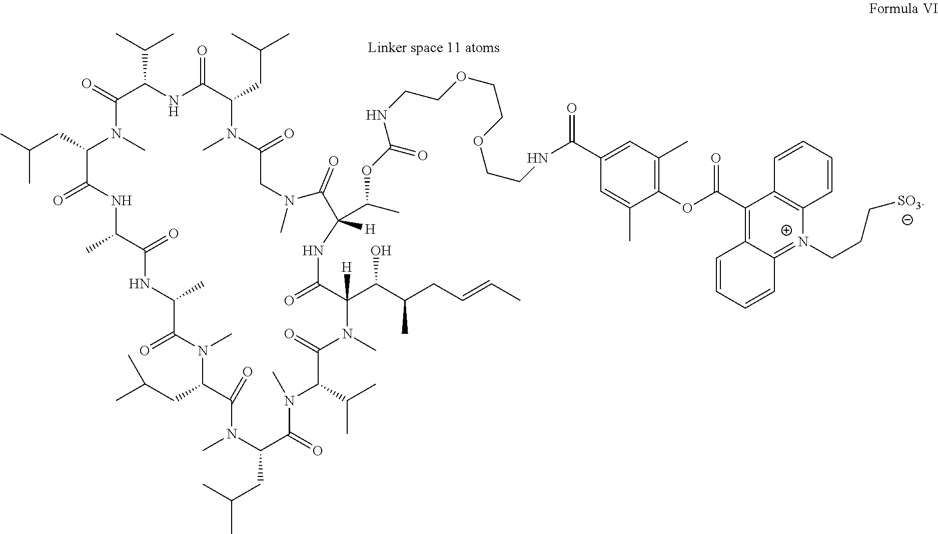

Illustrative embodiment 7. The composition of any one of illustrative embodiments 1-5, further defined as a Cyclosporine C-Acridinium Ester having the structure of Formula VII:

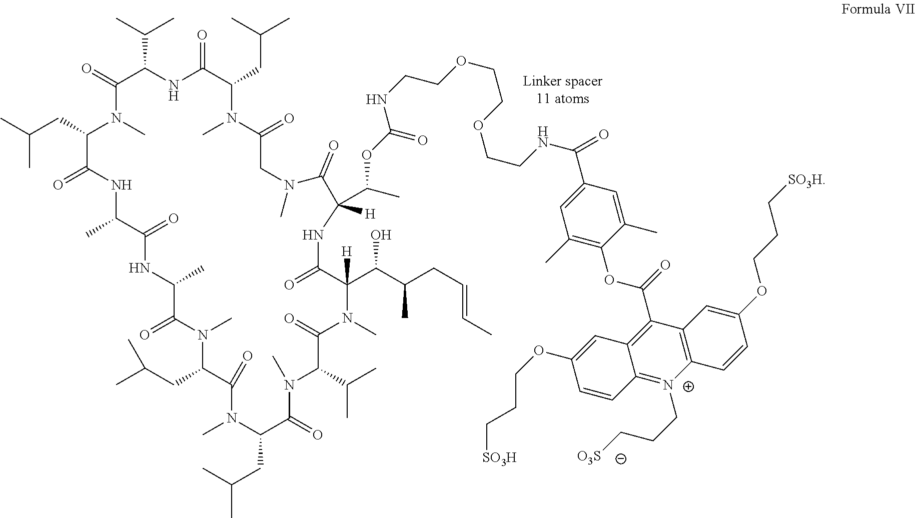

Illustrative embodiment 8. The composition of any of illustrative embodiments 1-5, further defined as a Cyclosporine C-Acridinium Ester having the structure of Formula VIII:

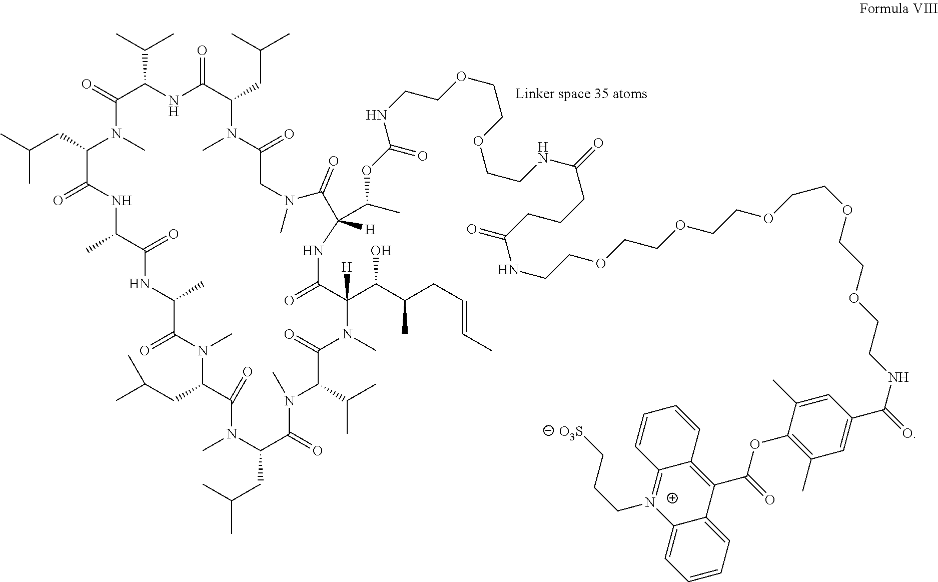

Illustrative embodiment 9. The composition of any of illustrative embodiments 1-5, further defined as a Cyclosporine C-Acridinium Ester having the structure of Formula IX:

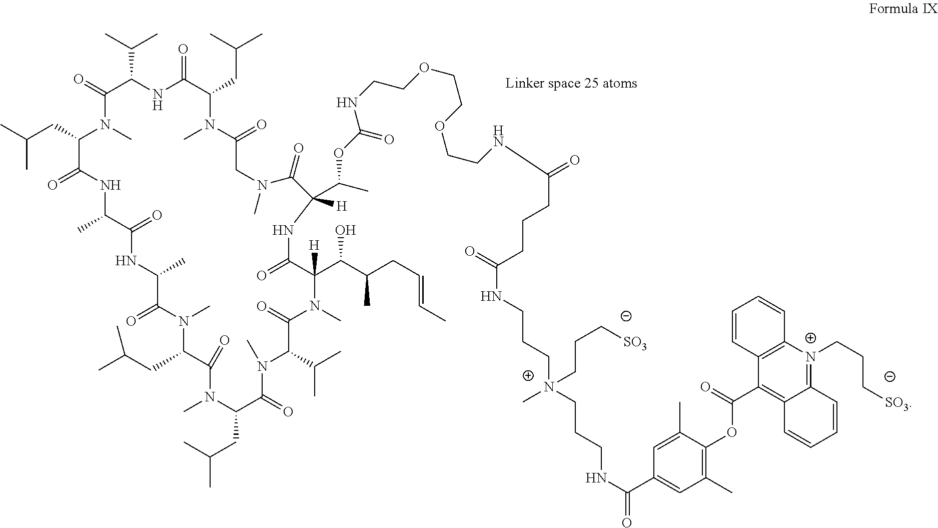

Illustrative embodiment 10. The composition of any of illustrative embodiments 1-5, further defined as a Cyclosporine C-Acridinium Ester having the structure of Formula X:

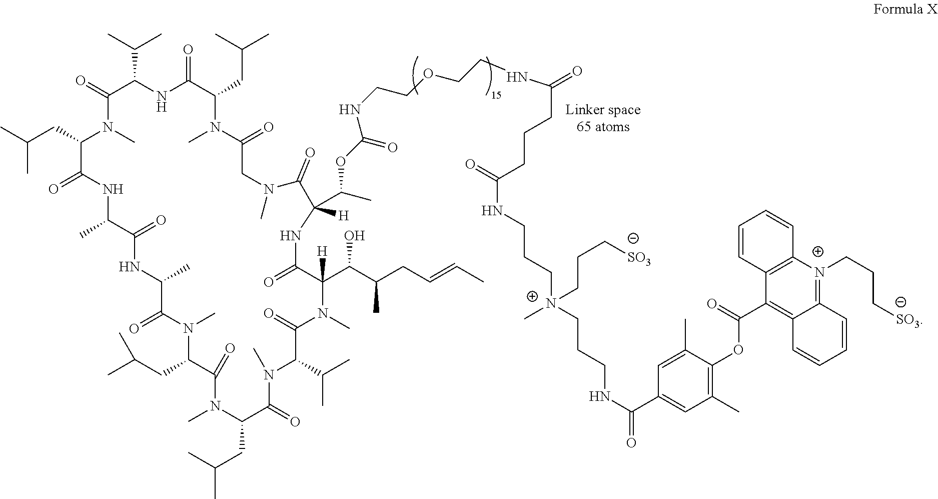

Illustrative embodiment 11. The composition of any of illustrative embodiments 1-5, further defined as a Cyclosporine C-Acridinium Ester having the structure of Formula XI:

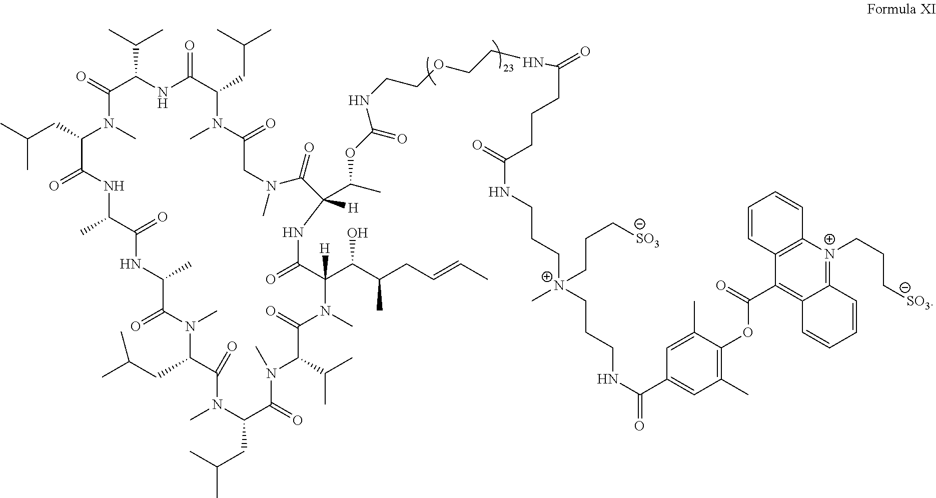

Illustrative embodiment 12. The composition of any of illustrative embodiments 1-5, further defined as a Cyclosporine C-Acridinium Ester having the structure of Formula XII:

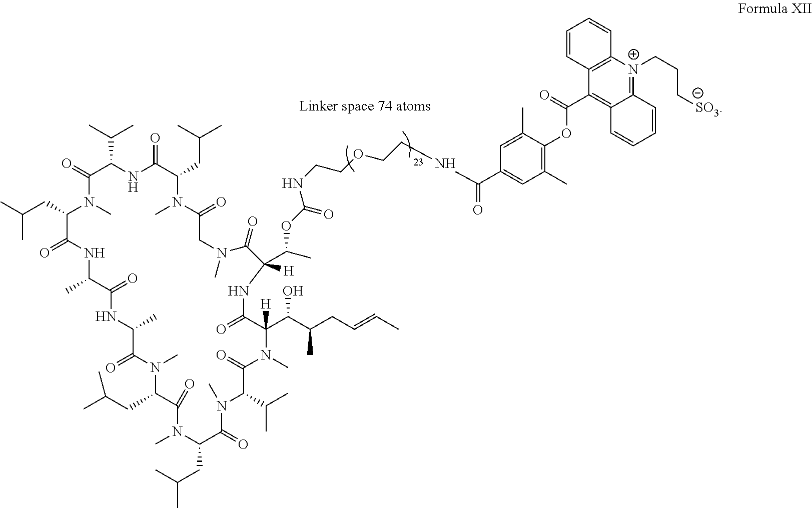

Illustrative embodiment 13. A composition, comprising: a cyclosporine A and an acridinium ester (A) linked via a spacer (B) and having the structure of Formula II:

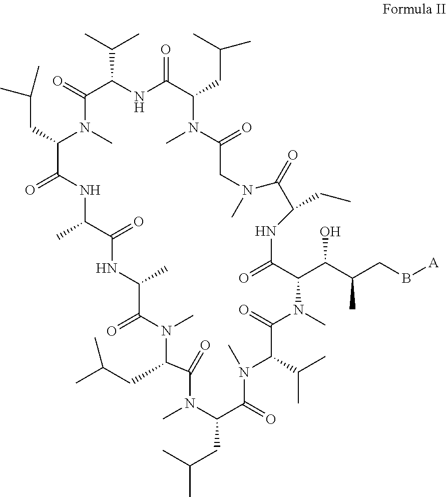

wherein: “A” comprises an acridinium ester; and “B” is a spacer having from about 5 atoms to about 100 atoms, each selected from the group consisting of C, H, O, N, S, and P atoms.

Illustrative embodiment 14. The composition of illustrative embodiment 13, wherein “A” of Formula II is an acridinium ester having the structure of Formula III:

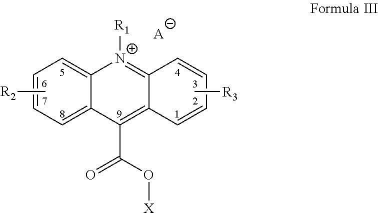

wherein:
“R₁” is selected from the group consisting of: an alkyl, alkenyl, alkynyl, or aralkyl group of 1 to 35 carbon atoms and 0 to 20 heteroatoms; a sulfopropyl or sulfobutyl group; and a group —R^{a}—Z, where R^{a} is a divalent radical selected from alkyl, alkenyl, alkynyl, aryl, or aralkyl group of 1 to 35 carbon atoms and 0 to 20 heteroatoms;
“R₂” is placed at one or more of positions C1 to C4, and each “R₂” is independently selected from the group consisting of hydrogen, alkyl, OR, OH, SR, SH, NH₂, and NR′R″, wherein R, R′, and R″ are each independently selected from the group consisting of an alkyl, alkenyl, alkynyl, aryl, and aralkyl group, wherein each group contains 0 to 20 heteroatoms;
“R₃” is placed at one or more of positions C5 to C8, and each “R₃” is independently selected from the group consisting of hydrogen, alkyl, OR, OH, SR, SH, NH₂, and NR′R″, wherein R, R′, and R″ are each independently selected from the group consisting of an alkyl, alkenyl, alkynyl, aryl, and aralkyl group, wherein each group contains 0 to 20 heteroatoms;
“X” is a group selected from a halogenated or unhalogenated, branched or straight-chained alkyl group; a substituted or unsubstituted aryl group; and a heterocyclic ring group; wherein the “X” group comprises 0 to 20 heteroatoms, and wherein the “X” group further comprises a functional group that links to the spacer “B” of Formula I; and
“A” is a counter ion selected from the group consisting of CH₃SO₃⁻, FSO₃⁻, CF₃SO₃⁻, C₄F₉SO₃⁻, CH₃C₆H₄SO₃⁻, a halide, CF₃COO⁻, CH₃COO⁻, and NO₃⁻.

Illustrative embodiment 15. The composition of illustrative embodiment 14, wherein “A” of Formula II is an acridinium ester having the structure of Formula IV:

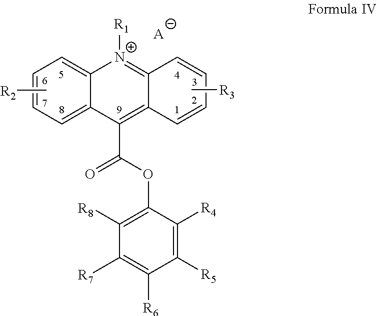

wherein: each of “R₄” and “R₈” is independently selected from hydrogen or an alkyl, alkenyl, alkynyl, alkoxyl (—OR), alkylthiol (—SR), or substituted amino group; each of “R₅,” “R₆,” and “R” is independently selected from hydrogen or an alkyl, alkenyl, alkynyl, aryl, or aralkyl group, wherein each group contains 0 to 20 heteroatoms; and one of “R₅,” “R₆,” and “R₇” further comprises a functional group that links to the spacer “B” of Formula II.

Illustrative embodiment 16. The composition of illustrative embodiment 15, wherein “A” of Formula II is a dimethylphenyl acridinium ester having the structure of Formula V:

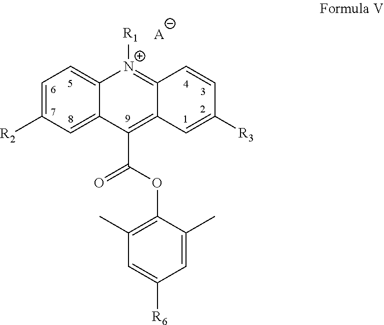

wherein: “R₁” is a methyl or a sulfopropyl group; each of “R₂” and “R₃” is independently selected from hydrogen or a methoxy, sulfopropyloxyl, or poly(ethylene)glycoloxy group; and “R₆” is an amide group (CONH—) connecting to the spacer “B” of Formula II.

Illustrative embodiment 17. The composition of illustrative embodiment 16, wherein R₁ is a sulfopropyl group, and wherein each of R₂ and R₃ is a hydrogen or a sulfopropyloxyl group.

Illustrative embodiment 18. The composition of any of illustrative embodiments 13-17, further defined as a Cyclosporine A-Acridinium Ester having the structure of Formula XIII:

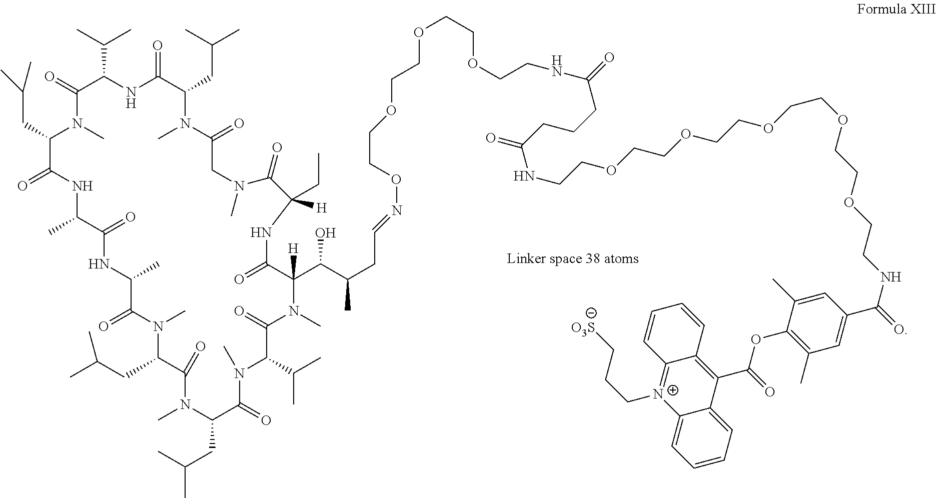

Illustrative embodiment 19. The composition of any of illustrative embodiments 13-17, further defined as a Cyclosporine A-Acridinium Ester having the structure of Formula XIV:

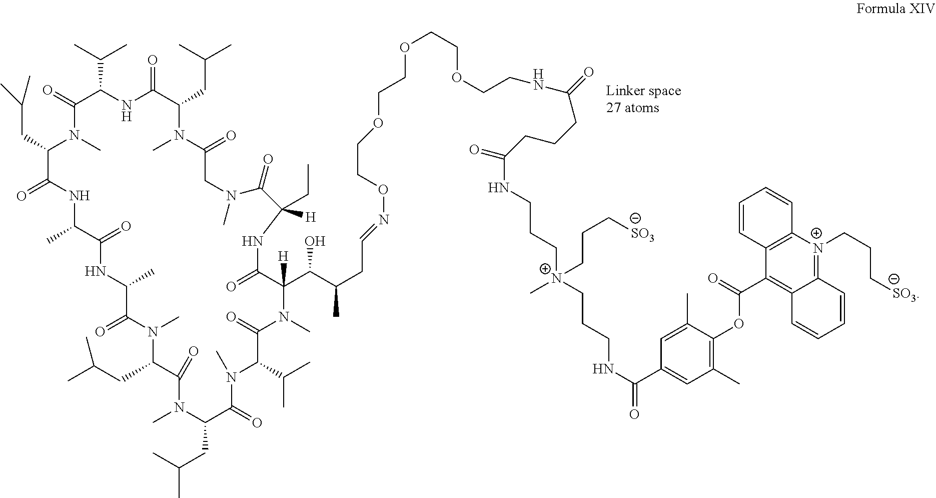

Illustrative embodiment 20. The composition of any of illustrative embodiments 13-17, further defined as a Cyclosporine A-Acridinium Ester having the structure of Formula XV:

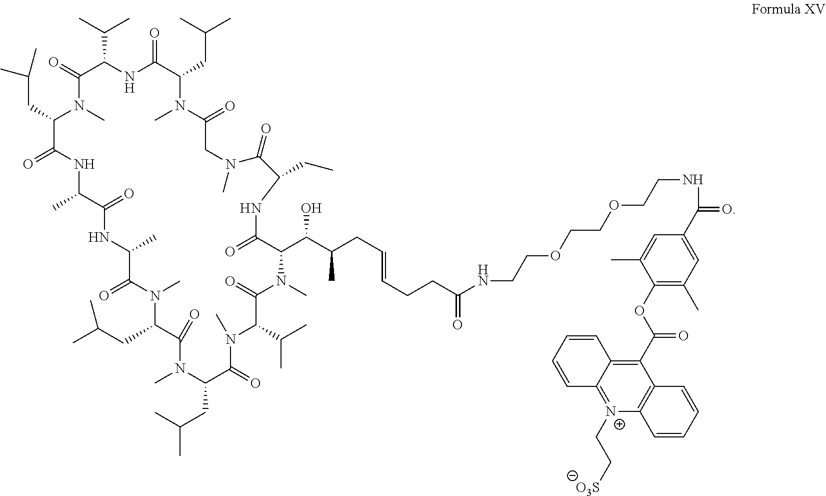

Illustrative embodiment 21. The composition of any of illustrative embodiments 13-17, further defined as a Cyclosporine A-Acridinium Ester having the structure of Formula XVI:

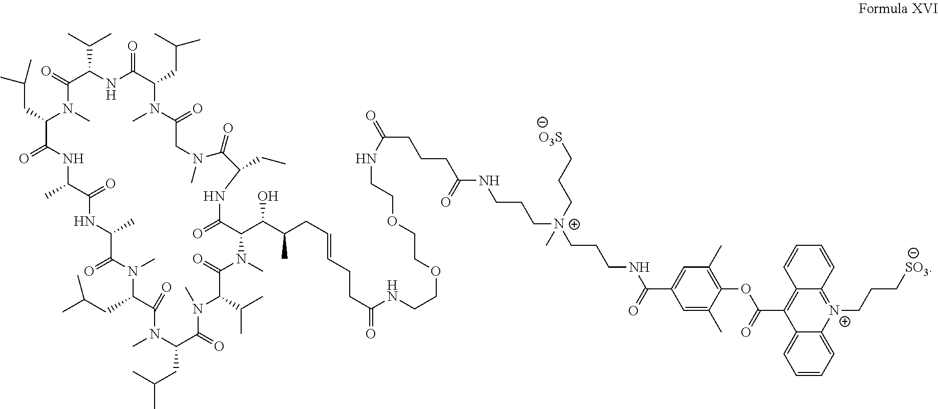

Illustrative embodiment 22. The composition of any of illustrative embodiments 13-17, further defined as a Cyclosporine A-Acridinium Ester having the structure of Formula XVII:

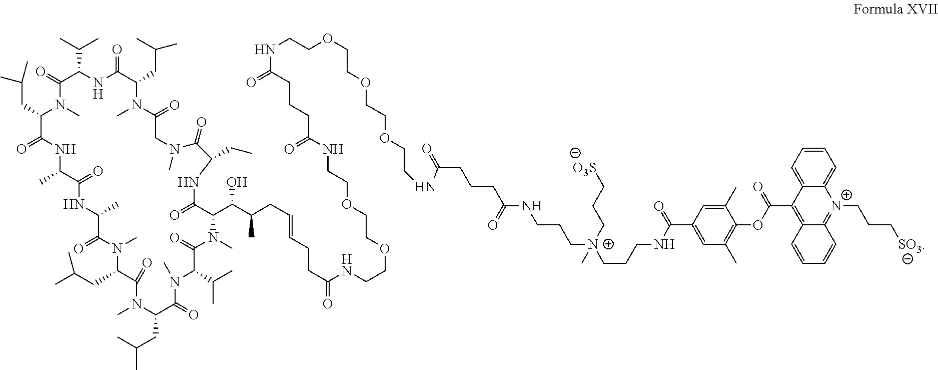

Illustrative embodiment 23. An immunoassay kit, comprising: a first reagent comprising the composition of any one of illustrative embodiments 1-22; and a second reagent comprising a solid phase having an antibody that specifically binds to Cyclosporine A directly or indirectly attached thereto.

Illustrative embodiment 24. The immunoassay kit of illustrative embodiment 23, further comprising at least one pretreatment agent.

Illustrative embodiment 25. A method of detecting Cyclosporine A in a sample, comprising the steps of: (a) combining, either simultaneously or wholly or partially sequentially, to form a mixture: (i) a sample suspected of containing Cyclosporine A; (ii) a first reagent comprising the composition of any one of illustrative embodiments 1-22; and (iii) a second reagent comprising a solid phase having an antibody that specifically binds to Cyclosporine A directly or indirectly attached thereto; (b) incubating the mixture under conditions that allow for binding of the antibody to Cyclosporine A present in the sample or to the first reagent, thereby forming a complex of Cyclosporine A/second reagent and/or a complex of first reagent/second reagent; and (c) detecting the complex formed of the first and second reagents; and (d) determining an amount of Cyclosporine A present in the sample based upon a reduction in the amount of first reagent/second reagent complexes formed when compared to an assay performed in the absence of sample.

Illustrative embodiment 30. The method of illustrative embodiment 29, further comprising the step of treating the sample with a pretreatment agent prior to step (a).

Thus, in accordance with the present disclosure, there have been provided compositions, kits, and devices, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

1. A composition, comprising:
a cyclosporine C and an acridinium ester linked via a spacer and having the structure of Formula I: 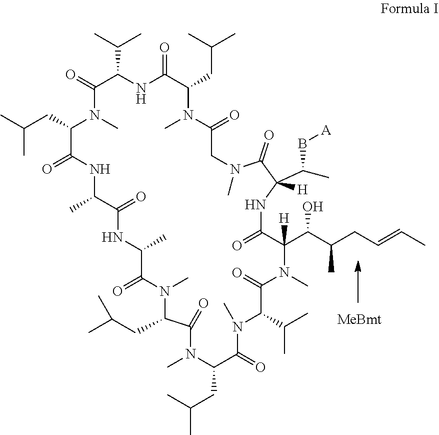
wherein:
“A” comprises an acridinium ester; and
“B” is a spacer having from about 5 atoms to about 100 atoms, each selected from the group consisting of C, H, O, N, S, and P atoms.

2. The composition of claim 1, wherein “A” of Formula I is an acridinium ester having the structure of Formula III: 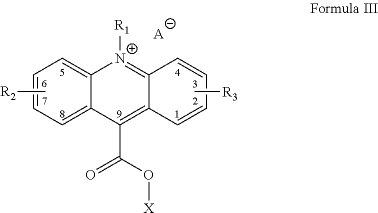
wherein:
“R1” is selected from the group consisting of:
an alkyl, alkenyl, alkynyl, or aralkyl group of 1 to 35 carbon atoms and 0 to 20 heteroatoms;
a sulfopropyl or sulfobutyl group; and
a group —R^{a}—Z, where R^{a} is a divalent radical selected from alkyl, alkenyl, alkynyl, aryl, or aralkyl group of 1 to 35 carbon atoms and 0 to 20 heteroatoms;
“R2” is placed at one or more of positions C1 to C4, and each “R2” is independently selected from the group consisting of hydrogen, alkyl, OR, OH, SR, SH, NH₂, and NR′R″, wherein R, R′, and R″ are each independently selected from the group consisting of an alkyl, alkenyl, alkynyl, aryl, and aralkyl group, wherein each group contains 0 to 20 heteroatoms;
“R3” is placed at one or more of positions C5 to C8, and each “R3” is independently selected from the group consisting of hydrogen, alkyl, OR, OH, SR, SH, NH₂, and NR′R″, wherein R, R′, and R″ are each independently selected from the group consisting of an alkyl, alkenyl, alkynyl, aryl, and aralkyl group, wherein each group contains 0 to 20 heteroatoms;
“X” is a group selected from a halogenated or unhalogenated, branched or straight-chained alkyl group; a substituted or unsubstituted aryl group; and a heterocyclic ring group; wherein the “X” group comprises 0 to 20 heteroatoms, and wherein the “X” group further comprises a functional group that links to the spacer “B” of Formula I; and
“A” is a counter ion selected from the group consisting of CH₃SO₃⁻, FSO₃⁻, CF₃SO₃⁻, C₄F₉SO₃⁻, CH₃C₆H₄SO₃⁻, a halide, CF₃COO⁻, CH₃COO⁻, and NO₃⁻.

3. The composition of claim 2, wherein “A” of Formula I is an acridinium ester having the structure of Formula IV: 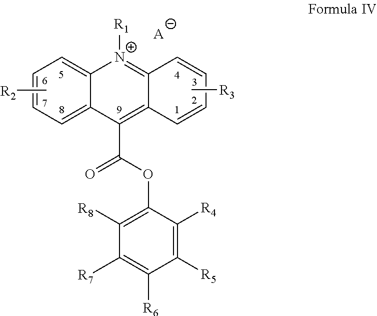
wherein:
each of “R₄” and “R₈” is independently selected from hydrogen or an alkyl, alkenyl, alkynyl, alkoxyl (—OR), alkylthiol (—SR), or substituted amino group;
each of “R₅,” “R₆,” and “R₇” is independently selected from hydrogen or an alkyl, alkenyl, alkynyl, aryl, or aralkyl group, wherein each group contains 0 to 20 heteroatoms; and
one of “R₅,” “R₆,” and “R₇” further comprises a functional group that links to the spacer “B” of Formula I.

4. The composition of claim 3, wherein “A” of Formula I is a dimethylphenyl acridinium ester having the structure of Formula V: 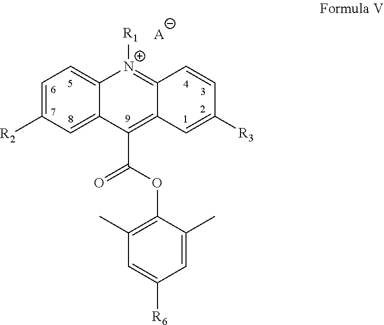
wherein:
“R₁” is a methyl or a sulfopropyl group;
each of “R₂” and “R₃” is independently selected from hydrogen or a methoxy, sulfopropyloxyl, or poly(ethylene)glycoloxy group; and
“R₆” is an amide group (CONH—) connecting to the spacer “B” of Formula I.

5. The composition of claim 4, wherein R₁ is a sulfopropyl group, and wherein each of R₂ and R₃ is a hydrogen or a sulfopropyloxyl group.

6. The composition of claim 1, further defined as a Cyclosporine C-Acridinium Ester having the structure of Formula VI:

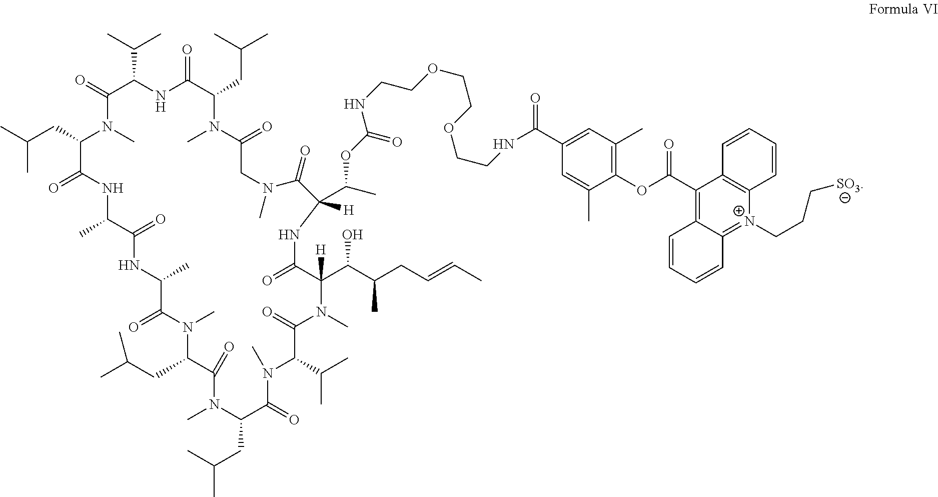

7.-22. (canceled)

23. An immunoassay kit, comprising:
a first reagent comprising the composition of claim 1; and
a second reagent comprising a solid phase having an antibody that specifically binds to Cyclosporine A directly or indirectly attached thereto.

24. The immunoassay kit of claim 23, further comprising at least one pretreatment agent.

25. A method of detecting Cyclosporine A in a sample, comprising the steps of:
(a) combining, either simultaneously or wholly or partially sequentially, to form a mixture:
(i) a sample suspected of containing Cyclosporine A;
(ii) a first reagent comprising the composition of claim 1; and
(iii) a second reagent comprising a solid phase having an antibody that specifically binds to Cyclosporine A directly or indirectly attached thereto;
(b) incubating the mixture under conditions that allow for binding of the antibody to Cyclosporine A present in the sample or to the first reagent, thereby forming a complex of Cyclosporine A/second reagent and/or a complex of first reagent/second reagent; and
(c) detecting the complex formed of the first and second reagents; and
(d) determining an amount of Cyclosporine A present in the sample based upon a reduction in the amount of first reagent/second reagent complexes formed when compared to an assay performed in the absence of sample.

26. The method of claim 25, further comprising the step of treating the sample with a pretreatment agent prior to step (a).
